SEQUENCE LISTING (57 sequences)

SEQ ID NO: 1 - protein, 511 aa, synthetic construct
MTESAEQDSGRFVLELPLRVHSGQDKTLRTRFEAGRQIYNACLGECLRRLDLMRESKDYQHARAMPRGKDRTKAFRDLDKRFEFRKYDLFHWTTQFTQSWLNQHVDSQTTKRLMERAFNATREYMFGKRGRPRFRGRRGLNSLESTSNTNGIKWRDGTIKWIGLEIPAIVDPEDPVQMHGLSSEIKYCRIVKRDLNGKRRWFIQLVLVGEPLQRFELGKGDVGLDIGPSTIAAVGTDDALLVQFCAELSEDQRETRRLQRKIDRQRRANNPDCYDGRGRAIKGKHPHKRSLRMRETEAQLAEVKRKLAAYRKSLHGELAHRILEMGDSIKTEKLSYKALQRIWGKSVGTRAPGMFVDLLRRKAESAGGEFVEFSTYHTRLSQTCHQCGAIVKKSLSVRWHNCDCGITAQRDLYSAFLASCVIEGSLDADLARERWSSAGPLLQTAFKETQLASSGASVPASFGLGQSQSESSVKRGQHATKVADVVTAQGIESARAAERLQNARESLLSDL

SEQ ID NO: 2 - protein, 749 aa, synthetic construct
MTMTTVKDIKQEHFQSKSFLDKDIKMAGRALAKYGEAACIEYLQNAPAAQGNLKRPTKCNIIAVSRPLAEWPALLASANIQKHVYSLTFSELEEFAEAFSKPSKGSRESILKVIETHGYSSAQGLNLMIGNAISTYKGVLKKVDNRNKKKAERIARINDSRKSNVNYVPLEFVPESALDDEGHLINPPGINRNLFCYQGLKLRPYAPTILPKEGEKTRHIHQKLNLPLAYDAYDRDPDAPIVCGALDRLSVPKGELGYVPEHPIGWKEPLLQRHNLKKKGRVRLGGNKSFKASQAIIVIISVGDDWVAFDARGLLRQIRRLNLCPKEEELTVETILSKFTDSPVINPNEGVVTFMVDPALVVGFGKKPVHRNSTKKRLLDLCSTSDVALVSVDLNKNNTFAYRISRVRKNGEELEPEIITFGCYDCSTKEFVSLRSAHDMAEEDYYNQALSSLSLEDQALIKASKNSDLVKAKVCERLSISKNDIDWDSMKYETSFISKFCSEHGIEDDDLLKIVERYKRGSRKGQIKSKKNAYDGKVIRKIVALPSDVNERLQKARWKFQRESSSFKRLAERKRQVANAYVNKVIVQAKKLSQCDVVAFVVEDGVNKFHGGGVREIGLDSLLRPKKEGRWLIQAFYRSITDLAPNKGIDVLMIDPRWTSTTCIVCGNVDKANRSSDNKEEFICTNTKCGDKRNADIEVATFNIQKCALTGRAMPGPEKRERSSDGKKSRTSRRTKAHQIIDEIHNAAE

SEQ ID NO: 3 - protein, 721 aa, synthetic construct
MPTKKKTYSSARKSSKVPTRNFVYGVGCKGEQPTPQDVIRDQLWEAHQYKVALIRNGLARIDAENAILCEVSPRLGELQRQSDDLTEKIKTMRAELKSPREDRADSPESAAIQAVVDERKAVYEEMKKVRKDLFALTSTYNADLNRKLEQGLLELAVEKDPAFAPLLDGCPEGENPKEFKKRVKRATSIIGPRNPKRVALNARLTEQMLSDDSVPDTWKRIKEVQQECHKKNLQARKDSKCFSGTYQAVDAAVRDAFQKSYHPRVPKFNRTGKVGIQLTGERTVNGKTVKGLPVSEVFSPDNQFSLELLPDHRVYGARKKHNRKEYPVMARARICVARGVRVETLVVMHRPLPEDGVIKWAYLEVKRVGLKTRYELRLVIEYFPKPRPKVTKKPLLAVNFGYRAMADGTIRLATTWDGEEVGGITLPSHIKRDEEFSRRLDGHADKRFNTAQKELGSWKKTVKDMPDELKEKIRNSGQWRAHGKLAVATHLLVRTYVPESETTVRRLWHTWRVERLEQGLDLFDERPFLENWFREQGVRSRNARFALYMEWWRRKDVHLVDLAKGFKARYKRRIKEIVRVQVARWRDSYEVIASNEWDMRKTARKPKPENDTRTKREINANGVRQIGGPGIVNLYLKEVFGDRRMTTDSKGASKIHFGCGGEGTGIETDLRCNKCGRGFDQDINAARHTWKEANGTLNGTPPKKAPKRKRSSRLAETTSSV

SEQ ID NO: 4 - protein, 686 aa, synthetic construct
MTARRQRSTEVRIYKYGVLAPSSDARSHLDEELFRANQYHNELIAIERTRRERFAAVRAEAVPELQGVEGRLMAIESELDVTFEAHRGGDDAASGRLAALKEERATLTARAKQLRDEFKALLAPGQQAFDERTSGAPAALLRELDRAGRALKEAKKIRRNPEVPILRRRLDELKRQRRSYLPSPHRKAEANETGLNQMMDEDWPDAWKRVARADAEAVVAIKKARAQKSVGAPTSRLIERAVEAVRRDSKMEPRFRRFRGEGQIGGQVPDADGTTKIFSAKNGLIRIGEKVSGSGRFAKRAVELRVGEWWIALSARWHRELPADARITWAWLKVRRDGWTKRYELQLTVESERHSTSRREVAQDGKVRVRLGWASIDDAPQVLRPGESPPDARYRPLIVATAEDERGTVTECVLPRSEIERLNHREANLRRISDLHYNETHKIFQSVVRRWLERHPESVPTWITERPVVRGEARRSMVDTMHLWRSHEALARASFRWCREVLGEERVNDLWLRWRDERKARGLDLFGRIGEFTRWARATGVVDSRELLVLYFEWWRRKELHLADWGTRQTRGAERARAQRYRLWGIDLSKLYATLTLEDIDLSRTAQDHGARDRAPNPVDSRVRSQQRDAAPSVMRAALLEVFGKDRTTILKRERSGGEDRPGTARRAEKKPGHERRESIDDRAAE

SEQ ID NO: 5 - protein, 499 aa, synthetic construct
MATKVYKYGARPVRCPLVDEQMRLGHRYYNALVEAENDRRKEGRGGDTPPAPPHDDCECKECREHWKILRKRMRDLPFIDVKPLRAEYAKKGLYWGTYLHVEKDFDAAKMKTNIFRTVKFRSWHRGGLIAVQIQRATHDRNGGKTMFQISDAPDSRSGRRNRGGRGRALVKMRVGSDGRVPVFCTPIKIERHRLMTGSPTWVKLTKRTDAERETWEVHVTCAGVEETPGAERGVVAVDCGWRKLPDGSWRLGYARGDQGAVSEFVLPATWSELAHRADRIRSHRDERLNALKAEDKRFSRCQSCLGVVRAVRKMREEGAPVDKDDEIKEWLRRDRHLWQYEVGCRRRSVARRRDEMRKWVRRLRDQYREVIVKDSNHKRMKESSGLHQAARRQGHHGSPGELVEALRQVFGADAMHVVSAVHTTNTCVECGHKNNFGPEREVICEGCGVVDDRDSVSTQNMIRLFADGQYSGPTARKTEAKFSKRHRTSEEGVSASPVA

SEQ ID NO: 6 - protein, 497 aa, synthetic construct
MKVYKYGITSDLDDQITAQMKAGHDYYNQLVEAENRRRYDLWFGGATPAPPHTGCSCKDCRDHYRNLTPPKPPHTPCTQKPCTACNAFWQPFNDASWLDLKPLRKQRPDGLYWGTYLLIERAFMQAVKTTKILDRVRFRPYRRSSTVCGVQIQKNKRLDAFFQIEARHDSRKGKRKGRRHRFRLRVGSQGRTPIWSDWINIELHRPWSGDIKWIAVVRAANGKCSLQITCNNENARTDNAAHGAVAVDISWRKLQNGSWRLAMARDLNGNLDELTLPPEWAELSARADRIRGHRDKRLNELAAVDSRFAVCKHPSGVERRARKLGIDQGDAIWDWVARDRHLDQYEAGCRRRSIARRRNDMRVWVRQLRRNYATVIIKDTSHKKLKEKKDLHQGARRQGQRAAPGEIVEQLRRVFGKGMQLVPAPMTNHVLCGPGHRAVEVGADLMLVCERCGARHDRDHVSTQNMLALWSAGEGVDPPARRTGGRFANQHGSSASP

SEQ ID NO: 7 - protein, 694 aa, synthetic construct
MGQLLNNTDRLIVVQRRSKPKSSWKIVARFATVAEAEVTKERYAADERREVEQIKNGEKKPDKKKKPQPSETAEYRVYYDTITRVYRYGLRSPDVERDVDQQMFQGHVYRNSLVAIYKASREEYRALLNTVGDVDAAGQHKREKAAIVEQLKQQIKTVRKRSRARAEPPELKARLKAAKAELTLATNSYKVLVAKADEAGLYTKGKKAIDATKGRAIKEARRVARTEQDLFWGTYGRREEFAARANKGHYDPKFHRWDYGDGSTWLAVQLQKGLSLSDLFSAKNRYLQIAPIDLLPDENDPRVWNKSVRKGERRRRSRTLVRVCIGSTSGKPVYATFPLELHRPLPANSKVKWAVVIRERVGRGQDCDKWSLHLSMEIPKKPVVRRPGVVGVNLGWRLKQSRKAGGVSILRVAYWADDQGNHGEVLLDDAIWHRDNKAKEGGYLGHARHLRSIRDKAFNAARDDLGSWLRGRRKRRQYIPKWLRTGAKNLGNWRSARRLAKLVWKWKDNRFADDVEIYRRLEGWRSIGADGKPFYEGWRKQDKHLHNWEAHSARRGRNKRKNTCQHFAKRLATLYGTVVVTQMDYAKAARRAGPGEEKESHDAARRQRQVAAPSRLRDAIQNACSTHGSDFNALPVRTDWCHVCGTPNLWASEKSKIEDIKHTCKGCGNVIDQDHNAAEIVWRIWTDNFNKKAA

SEQ ID NO: 8 - protein, 633 aa, synthetic construct
MAKKHKNQSKATRVYAFGAYPSVENSELVDDIFYRAWRFRNDLIRVELDYRKRRDEIVREYSLKVRNLEAEHQCLTQEIADREGEVNTQSIQDRKRCLAPELKGLRKQLKQTEYALVKEQRAEERRLKQYYSSLTNERLEGLQQQMTAAKSPATKGKLNKAIRAERESIWQEAGINADMMVAEYEASDRNATLDTESGLLWPTKNTIKADVARAIKDGPRIRGYDGGGRIGGQPSTKGISVAEALAGTSNWLRITGPHEKCTRGHHNRLNTAWIRIDSEGRNPVWAVIPFVMHRPLPADAHITNVYLVRNRVASKFKYEVHFVLAAESWVKDDLAAEGECAIDLNWRWMDGSLRVASWGGMDGEEGDLFLPAKVVRMFEQVNEIKSIRDREFNIVRDRLAAWIKSHEVPEWLQERGKFLSRWRSQRKLAKLVLYWRDRRFDGDDEIFAALDGTAERVQFHGKRDKAAWNGWRNWDKHLYEWERNLHQKAIRCRQAIYRDFAAELTRRYHNIVMEDINWHEIGKRAEVGEADNPTVRAKKPIGAVGELAQYIIQRAACPEVVEPQNTTRRCPVCRTIGKSGPALYHECKHCGRVWDQDKKAWLNMLLAGERLDARLTKKKRDSLRKAHREERAA

SEQ ID NO: 9 - protein, 556 aa, synthetic construct
MVARKTSTLPTKIWSSAAKCPAVSLEKRDEILSRAHAYTHRLILIEQSRLTEYRATRKHYAPEIEELQDALQDALAWRDEDAKQQAIDSARGALRTAIAKFEADQCGAARAEYAKRRAAACAGMGPAIKARLNNEILVAMLAEDTWPDSWKDLAMADRDSQDQVKRARSECGLTKGCYLAVENAVNAAIKDSRPLAPQLPPWDGSGRIAVQIENNNRWADIVAGRNRFLRVEYTDDRPLGSCGNSSIRCWAVFIRVGSNSDRTPVWLEFSARLHRHPPDDAIVKWAWIKCYKSAGREQHRFQLTFEHESLRWLPKPGAVGSLDLQCCHEPVAAGVRVARWCGSDGLSGEVVVPDDVLGNFRFAHGIRSALDGVRAHGYRVVRKWLALGGNHVTGWRRLSKDWRRAQMLAVCQSYVDFHETKSPNDSYLPLHRSMLTRDVSAAQDRRFALWLDSWLRKERHLWRYWRDSEARAVNLRNTLFLQAAHELRRKYVSLSSDTSHLIANPKMSQTERASRQAVAPGECRAIFREAFGLSRAPGSRGTSKAPKKRRRSQSGA

SEQ ID NO: 10 - protein, 721 aa, synthetic construct; identical to SEQ ID NO: 3

SEQ ID NO: 11 - protein, 486 aa, synthetic construct
MAEKQKEVIITRKIEVHLHHDPVADPDKVEYNRHWEVWHTINNNLYKAANRIVSHKFFNDEYEHRLQLHSPEFQEINRKFARAKKEQLSAEEIADLRSRIKELYAQFANEKRTFLRGGAAVGPNPEQNSTYKVVSDEFGKLVPSEILTCLNQSVASTYSEYKKQITRGERTLPNYKKGMPVPIPMKAAGKLRLRRRDDNSIYVLTAGGLEWDLSFGRDRSNNREIVERVLSGQYDVGGSGIQEKGRKIFLLLVVKLPASQNELNPERVVGVDLGLNIPLYAALNDNEYGGLSIGSRDKFMKERERMSARRRELQRSLRISTQGGHGRSHKLKALDQFEEKERNWVHTQNHIFSRQVVEYAKSNGAGVIQMESLKGFGKDKEEHIEQGFKYVVRYWSYFELQTLIQQKAEREGIEVRKVNPYHTSQTCSFCGHYEKGQRVSQSTFICKNPACAHGKGKQLKDGTFAGINADWNAARNIALSTEWVKE

SEQ ID NO: 12 - protein, 486 aa, synthetic construct
MAAEFTCITRKIEVHLHRHGEGDEAEMRLKEEYRIWDEINDNLYKAANRVVSHCFFNDSYEYRLKLHSPRFQEIEKLLKNAKRNKLSNDDIRELKEERSLLFSDFKKQRLAFLQGNSGKGSEQNSTYNVISNEFVDVIPSEVLTNLNQDISSTYKNYALEVERGARTIPNYKKGIPVPFSIKRNGELRLRKREDGSVYITFPRGLEWDLNFGRDRSNNREIVERVLSGLYDVGNSSIQETKSRKRFLLLVVKIPKEASKLDTNKVVGVDLGVAVPLYAALNDNQYGGMSIGSYEQFTKVRQRMFAQKKELQRSLRHTTNGGHGRKQKLQALERLEGKERNWVHLQNHIFSKSLIEYALGNKAGVVQMERLTGFGRDVNDELGEGYKFVLRYWSFFELQTMIEYKAKAAGIEVRYINPYHTSQTCSFCGHYEKGQRINQSTFICKNPDCIKGKGERKSDGSYKGINADWNAARNIALSKEYVEKKKK

SEQ ID NO: 13 - protein, 616 aa, synthetic construct
MAFKKRNDDTHYKVYRYGAILRPKIDGEKNPDFLLIQDQLRQANEYYNVLVDIETDRRNEWNAIIDADKDVIKAKSAVEEACDRVEAAERALKDARCKTGKRRPDRKLAQAVSREKRKRTKARKAVDSAKERAKQKKKKVLDRHKMKFKKIFNEARSEAISKGLYWGTAAVTLDALPQSGPPPSKRPLYGDGQIAVQIQCKSIPDKYFQIHDQEGWRSKRPGKLLYIRVASKNKAGEITNHGGHTPVWAKVPFLLDRPLPENAPVKWIQVVKKTVGMTKHWSISFTVELPGKRSIVEKVEKTQCRGKVIAFDLGWRKNADGSIRVAYGTDSSGKSFEITIPAWLSNGWDRVQYIRSVRDKMFDQIRDELHEWRWGRKIPDWLTEKLKFSYNWKAQKRLGALLGHWRDHRFEGDASMVAKIEKWDKKDRHLLKYERYHERQCIHQRRELYRHWARWLAKNYDAVVFEDLNFAELYKKPDIESEKDGVIGLHRRASPGILRATIEYAFTDEKKRAVRVKHEGSSQTCSSCWEITMNVGKEVKAKCEACGYEADRDCNASRVLLARGGVVRTSPGRRTKPKAQWYERLMAVPLRVREGQEKLGRNHSQSDNQGADFVTV

SEQ ID NO: 14 - protein, 569 aa, synthetic construct
MYGHASDPSRVYAYGAQPPRGEDGDLIRAQIDAGWRYRNDLVALELDRRERVGEVVRAHYPDVADQDAAIEETEAEIEDARAAIRAANQRKRARDPGPGRARTIREMRDRLANLRREHRAARKRAYADDAVQADLAVVDTGARTEQKRLRAESGLHWGSYLAVEQSLSGIRSGAPPRFARRATWGGQVAVQVQGGATWEELTEGHGQFRVEVEPLPPNATPGGRRSKRPYCRAYFRVGSEGPGNRTPVWAMVRFVLHRPIPDDGHVKWVRLTRQRVGTDEKWQVQFVVAREEWVHHDTGDASGVCGVDLGWRTLDDGSLRVAYAMGEDGESEELVIPAERVGALDKADSLRAIRDREFDLIRAQLEHWLATEPDLPEWLAEDTRTLGQWRAQARLAAVVIRWRDQRFEGDDDIYESIEAWRKQDKHLYDWEGASRAKFQRWRKQHYERFAARLRWRYETVIVEDADWAQMARTPPAEEHDEPERLNRRRMARIASPGYLREALEHGHPDVRRAPSANTTRTCSVCGCDPAGDWDPITELDYRCEAGHVMDQDCNAAINLMHASGDVVRE

SEQ ID NO: 15 - protein, 642 aa, synthetic construct
MSNAIASHFASMILRAYKTELDPTVEQRRALARHVAGARVAHNWVLERWRLLAGLPRLVSFLRALDGISAAREKQAFEAWRLGFILHALLCGERRTIRRPKRTKKGGVTEYTEKIVFSARPPPGPTLDQSPDSDRIHAQLTAEKRIDESTLAWLTSLSSFAVREGAGDVGSGYKAYFRRLKKHLKGNHSECKPRRHRRGCQLGEPKFRSARERRYHVDQPNPIRITARTILLPGIGEVRLKERGYLPETATTLDAKGHSKGSHRLKQGGKVCGVGISERDGRWRVSVRAEVPFPRVQPRGCGNALRWCVVPRISGRKIGVENGVRVLVATYDGDTASTFVDSGLRDDERIERFVRTRKLWERRMARRWREGVAVRDQSTGWREAKRRVRHYHARIVDLRDDRVGKAVRRIVDSGAEVALLREPHVADLLNRETAPDARVRNRLAPDVHGARMGDVRRRLEYKMLWARGRVERVDRFEPVTKRCSECGAVRATSPAYPDFRCAQCGHVEDRDDANAPKNLFGYDSSGSSSSGEAGPRSAGSKAPEGVSNGREKRSARVEGQPSATAPYRSGNRARPGAPISDGPIGPIFHSESENEQSCAAQPCAEQFRSATASPLADEADRSQTILQARERSTRSLDESKSK

SEQ ID NO: 16 - DNA, 235 bp, synthetic construct
  source 1..235: mol_type = other DNA
gcccgtaccgaaagccccctcctggaagcctacgggttgttgttcacaagcatggttggagggcgaggttgaagcgggtgatgtggcggcggagccttcgttgtcgggggaccttcagggagagggcggttcgatgtggcaggtgtgggagtaagattgggttgcttcgcactcggttctctcaggatgaggtcgagttgctttccatccacttgcttcacatccggcgggggtg

SEQ ID NO: 17 - DNA, 612 bp, synthetic construct
  source 1..612: mol_type = other DNA
agtatctacaccagaagagcaagctcgtgagattctcgatgcggctggggtgacctacgatcgggtctatgtctacgatgggtgcacgcaagagatttgcgggcccatgccccgggtcattgttcggcgggagggttccgttcccgtgtgttacagtatacctgaactcagggaagcggtgggggagtggaagacgaggtgggaacgaatttcgtcgagggattgaatttccttggaggcagatcgtagtagggggtgaacgacgggacgaaaggaggccgagtggcctacaaaatggtgagactcttcgatggctaccagctcaaccacccgtgcgaaacgtttcaagggtggctcacgacctcaaatggtgagactggctactacggctaccgccgtgacgccaaaaaggactcccggaaggcacgtcgtgctaatgacaaggccgcttgccgggactattgaggcccccttttgggggcctcttgctgtttgtgccgccacatcttgccgtcgatgcggccatccacggcgggttcgagctgggagtagccgagggcggggccctcatcgtagtaggggtgaacaacgcaacgaggtgtacgaaagatc

SEQ ID NO: 18 - DNA, 239 bp, synthetic construct
  source 1..239: mol_type = other DNA
ttgtcccatgtttcaagggtggctcacgacctcaaatggtgagactatctgcgatcggtctagtggtcctccagtgccggggacctctcgcaccgcccccggctctttgaaatccttggtgttttacggcgttcggtgttcaagtagggtgcatctaacgttcggagatgtcctcgatgaggggacctctcgcaaagcaggctctaagtactcgaatcctggtggattccacgggacct

SEQ ID NO: 19 - DNA, 278 bp, synthetic construct
  source 1..278: mol_type = other DNA
gaagtgccattccgcctgacctgcccgtaccgaaagccccctcctggaagcctacgggttgttgttcacaagcatggttggagggcgaggttgaagcgggtgatgtggcggcggagccttcgttgtcgggggaccttcagggagagggcggttcgatgtggcaggtgtgggagtaagattgggttgcttcgcactcggttctctcaggatgaggtcgagttgctttccatccacttgcttcacatccggcgggggtgaggctaggtggaggctcagtg

SEQ ID NO: 20 - DNA, 655 bp, synthetic construct
  source 1..655: mol_type = other DNA
gaagtgccattccgcctgacctagtatctacaccagaagagcaagctcgtgagattctcgatgcggctggggtgacctacgatcgggtctatgtctacgatgggtgcacgcaagagatttgcgggcccatgccccgggtcattgttcggcgggagggttccgttcccgtgtgttacagtatacctgaactcagggaagcggtgggggagtggaagacgaggtgggaacgaatttcgtcgagggattgaatttccttggaggcagatcgtagtagggggtgaacgacgggacgaaaggaggccgagtggcctacaaaatggtgagactcttcgatggctaccagctcaaccacccgtgcgaaacgtttcaagggtggctcacgacctcaaatggtgagactggctactacggctaccgccgtgacgccaaaaaggactcccggaaggcacgtcgtgctaatgacaaggccgcttgccgggactattgaggcccccttttgggggcctcttgctgtttgtgccgccacatcttgccgtcgatgcggccatccacggcgggttcgagctgggagtagccgagggcggggccctcatcgtagtaggggtgaacaacgcaacgaggtgtacgaaagatcaggctaggtggaggctcagtg

SEQ ID NO: 21 - DNA, 282 bp, synthetic construct
  source 1..282: mol_type = other DNA
gaagtgccattccgcctgacctttgtcccatgtttcaagggtggctcacgacctcaaatggtgagactatctgcgatcggtctagtggtcctccagtgccggggacctctcgcaccgcccccggctctttgaaatccttggtgttttacggcgttcggtgttcaagtagggtgcatctaacgttcggagatgtcctcgatgaggggacctctcgcaaagcaggctctaagtactcgaatcctggtggattccacgggacctaggctaggtggaggctcagtg

SEQ ID NO: 22 - DNA, 128 bp, synthetic construct
  source 1..128: mol_type = other DNA
taatacgactcactataggctaaagccaatgatgattgctctttacgaagagactggagatatcttgaaccttgcatccccggaagagagtcgctaaagccaatgatgattgctctttacgaagagac

SEQ ID NO: 23 - DNA, 171 bp, synthetic construct
  source 1..171: mol_type = other DNA
gaagtgccattccgcctgaccttaatacgactcactataggctaaagccaatgatgattgctctttacgaagagactggagatatcttgaaccttgcatccccggaagagagtcgctaaagccaatgatgattgctctttacgaagagacaggctaggtggaggctcagtg

SEQ ID NO: 24 - DNA, 126 bp, synthetic construct
  source 1..126: mol_type = other DNA
taatacgactcactataggtttcaagggtggctcacgacctcaaatggtgagacgtcgaggcttgcgacgtggtggctcacggaagacgagtttcaagggtggctcacgacctcaaatggtgagac

SEQ ID NO: 25 - DNA, 126 bp, synthetic construct
  source 1..126: mol_type = other DNA
taatacgactcactataggtctcaccatttgaggtcgtgagccacccttgaaacgtcgaggcttgcgacgtggtggctcacggaagacgagtctcaccatttgaggtcgtgagccacccttgaaac

SEQ ID NO: 26 - DNA, 169 bp, synthetic construct
  source 1..169: mol_type = other DNA
gaagtgccattccgcctgaccttaatacgactcactataggtttcaagggtggctcacgacctcaaatggtgagacgtcgaggcttgcgacgtggtggctcacggaagacgagtttcaagggtggctcacgacctcaaatggtgagacaggctaggtggaggctcagtg

SEQ ID NO: 27 - DNA, 169 bp, synthetic construct
  source 1..169: mol_type = other DNA
gaagtgccattccgcctgaccttaatacgactcactataggtctcaccatttgaggtcgtgagccacccttgaaacgtcgaggcttgcgacgtggtggctcacggaagacgagtctcaccatttgaggtcgtgagccacccttgaaacaggctaggtggaggctcagtg

SEQ ID NO: 28 - DNA, 61 bp, synthetic construct
  source 1..61: mol_type = other DNA
taatacgactcactatagtgatgattgctctttacgaagagactggagatatcttgaacct

SEQ ID NO: 29 - DNA, 61 bp, synthetic construct
  source 1..61: mol_type = other DNA
taatacgactcactatagtgatgattgctctttacgaagagacgtcgaggcttgcgacgtg

SEQ ID NO: 30 - RNA, 43 nt, synthetic construct
  source 1..43: mol_type = other RNA
tgatgattgctctttacgaagagactggagatatcttgaacct

SEQ ID NO: 31 - RNA, 43 nt, synthetic construct
  source 1..43: mol_type = other RNA
tgatgattgctctttacgaagagacgtcgaggcttgcgacgtg

SEQ ID NO: 32 - DNA, 174 bp, synthetic construct
  source 1..174: mol_type = other DNA
taatacgactcactatagtgcaccctacttgaacaccgaacgccgtaaaacaccaaggatttcaaagagccgggggcggtgcgagaggtccccggcactggaggaccactagaccgatcgcagatagtctcaccatgaaaatggtgagactggagatatcttgaaccttgcatc

SEQ ID NO: 33 - DNA, 186 bp, synthetic construct
  source 1..186: mol_type = other DNA
taatacgactcactatagtgcaccctacttgaacaccgaacgccgtaaaacaccaaggatttcaaagagccgggggcggtgcgagaggtccccggcactggaggaccactagaccgatcgcagatagtctcaccatttgagggaaacctcaaatggtgagactggagatatcttgaaccttgcatc

SEQ ID NO: 34 - RNA, 156 nt, synthetic construct
  source 1..156: mol_type = other RNA
tgcaccctacttgaacaccgaacgccgtaaaacaccaaggatttcaaagagccgggggcggtgcgagaggtccccggcactggaggaccactagaccgatcgcagatagtctcaccatgaaaatggtgagactggagatatcttgaaccttgcatc

SEQ ID NO: 35 - RNA, 168 nt, synthetic construct
  source 1..168: mol_type = other RNA
tgcaccctacttgaacaccgaacgccgtaaaacaccaaggatttcaaagagccgggggcggtgcgagaggtccccggcactggaggaccactagaccgatcgcagatagtctcaccatttgagggaaacctcaaatggtgagactggagatatcttgaaccttgcatc

SEQ ID NO: 36 - protein, 7 aa, Simian virus 40
PKKKRKV

SEQ ID NO: 37 - protein, 16 aa, unidentified
KRPAATKKAGQAKKKK

SEQ ID NO: 38 - protein, 9 aa, unidentified
PAAKRVKLD

SEQ ID NO: 39 - protein, 11 aa, unidentified
RQRRNELKRSP

SEQ ID NO: 40 - protein, 38 aa, Homo sapiens
NQSSNFGPMKGGNFGGRSSGPYGGGGQYFAKPRNQGGY

SEQ ID NO: 41 - protein, 42 aa, unidentified
RMRIZFKNKGKDTAELRRRRVEVSVELRKAKKDEQILKRRNV

SEQ ID NO: 42 - protein, 8 aa, unidentified
VSRKRPRP

SEQ ID NO: 43 - protein, 8 aa, unidentified
PPKKARED

SEQ ID NO: 44 - protein, 8 aa, unidentified
PQPKKKPL

SEQ ID NO: 45 - protein, 12 aa, Mus sp.
SALIKKKKKMAP

SEQ ID NO: 46 - protein, 5 aa, unidentified
DRLRR

SEQ ID NO: 47 - protein, 7 aa, unidentified
PKQKKRK

SEQ ID NO: 48 - protein, 10 aa, Hepatitis delta virus
RKLKKKIKKL

SEQ ID NO: 49 - protein, 10 aa, Mus sp.
REKKKFLKRR

SEQ ID NO: 50 - protein, 20 aa, Homo sapiens
KRKGDEVDGVDEVAKKKSKK

SEQ ID NO: 51 - protein, 17 aa, Homo sapiens
RKCLQAGMNLEARKTKK

SEQ ID NO: 52 - DNA, 32 bp, synthetic construct
  source 1..32: mol_type = other DNA
gttggatataccaacaattttacgaccggtta

SEQ ID NO: 53 - DNA, 36 bp, synthetic construct
  source 1..36: mol_type = other DNA
gtttcaagggtggctcacgacctcaaatggtgagac

SEQ ID NO: 54 - DNA, 36 bp, synthetic construct
  source 1..36: mol_type = other DNA
gtcgaagtcgaaatgacggcttggatctcttgtcag

SEQ ID NO: 55 - DNA, 36 bp, synthetic construct
  source 1..36: mol_type = other DNA
gtcgcaaagaccatctggacggtcagagatccagag

SEQ ID NO: 56 - DNA, 36 bp, synthetic construct
  source 1..36: mol_type = other DNA
gtctcaccgtagtgagcaatttttatcagtcgttac

SEQ ID NO: 57 - DNA, 29 bp, synthetic construct
  source 1..29: mol_type = other DNA
ggtcgacccatgcacgcagggatgacccg

(19) United States
(12) Patent Application Publication
(10) Pub. No.: US 2025/0002881 A1
(43) Pub. Date: Jan. 2, 2025

(54) CLASS II, TYPE V CRISPR SYSTEMS

(71) Applicant: Metagenomi, Inc., Emeryville, CA (US)

(72) Inventors: Brian C. THOMAS, Berkeley, CA (US); Christopher BROWN, Albany, CA (US); Cindy CASTELLE, San Francisco, CA (US); Lisa ALEXANDER, Albany, CA (US); Liliana GONZALEZ-OSORIO, Hercules, CA (US); Paula MATHEUS CARNEVALI, Pinole, CA (US); Dom CASTANZO, Oakland, CA (US)

(21) Appl. No.: 18/596,189

(22) Filed: Mar. 5, 2024

Related U.S. Application Data
(63) Continuation of application No. PCT/US2022/075988, filed on Sep. 6, 2022.
(60) Provisional application No. 63/241,932, filed on Sep. 8, 2021.

(51) Int. Cl. C12N 9/22; C12N 15/11

(52) U.S. Cl. CPC C12N 9/22; C12N 15/111; C07K 2319/09; C12N 2310/20

(57) ABSTRACT

Described herein are methods, compositions, and systems derived from uncultivated microorganisms useful for gene editing.

### CROSS-REFERENCE

This application is a continuation of International Application No. PCT/US2022/075988, entitled “CLASS II, TYPE V CRISPR SYSTEMS”, filed on Sep. 6, 2022, which claims the benefit of U.S. Provisional Application No. 63/241,932, entitled “CLASS II, TYPE V CRISPR SYSTEMS”, filed on Sep. 8, 2021, each of which is incorporated herein by reference in its entirety.

### SEQUENCE LISTING

The instant application contains a Sequence Listing which has been submitted electronically in XML format and is hereby incorporated by reference in its entirety. Said XML copy, created on Feb. 20, 2024, is named 55921-732_302_SL.xml and is 63,199 bytes in size.

### BACKGROUND

Cas enzymes along with their associated Clustered Regularly Interspaced Short Palindromic Repeats (CRISPR) guide ribonucleic acids (RNAs) appear to be a pervasive (˜45% of bacteria, ˜84% of archaea) component of prokaryotic immune systems, serving to protect such microorganisms against non-self nucleic acids, such as infectious viruses and plasmids by CRISPR-RNA guided nucleic acid cleavage. While the deoxyribonucleic acid (DNA) elements encoding CRISPR RNA elements may be relatively conserved in structure and length, their CRISPR-associated (Cas) proteins are highly diverse, containing a wide variety of nucleic acid-interacting domains. While CRISPR DNA elements have been observed as early as 1987, the programmable endonuclease cleavage ability of CRISPR/Cas complexes has only been recognized relatively recently, leading to the use of recombinant CRISPR/Cas systems in diverse DNA manipulation and gene editing applications.

### SUMMARY

In some aspects, the present disclosure provides an engineered nuclease system comprising: (a) an endonuclease comprising a RuvC domain, wherein the endonuclease is derived from an uncultivated microorganism, and wherein the endonuclease is not a Cas12a endonuclease; and (b) an engineered guide RNA, wherein the engineered guide RNA is configured to form a complex with the endonuclease and the engineered guide RNA comprises a spacer sequence configured to hybridize to a target nucleic acid sequence. In some aspects, the present disclosure provides an engineered nuclease system comprising: (a) an endonuclease having at least 75% sequence identity to any one of SEQ ID NOs: 1-15 or a variant thereof; and (b) an engineered guide RNA, wherein the engineered guide RNA is configured to form a complex with the endonuclease and the engineered guide RNA comprises a spacer sequence configured to hybridize to a target nucleic acid sequence. In some embodiments, the endonuclease comprises a RuvCI, II, or III domain. In some embodiments, the endonuclease has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% identity to a RuvCI, II, or III domain of any one of SEQ ID NOs: 1-15 or a variant thereof. In some embodiments, the RuvCI domain comprises a D catalytic residue. In some embodiments the RuvCII domain comprises an E catalytic residue. In some embodiments the RuvCIII domain comprises a D catalytic residue. In some embodiments, the RuvC domain does not have nuclease activity. In some embodiments, the endonuclease further comprises a WED II domain having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% identity to a WED II domain of any one of SEQ ID NOs: 1-15 or a variant thereof. In some embodiments, the guide RNA comprises a sequence with at least 80% sequence identity to the non-degenerate nucleotides of any one of SEQ ID NOs: 30-35. In some aspects, the present disclosure provides an engineered nuclease system comprising: (a) an engineered guide RNA comprising a sequence with at least 80% sequence identity to the non-degenerate nucleotides of any one of SEQ ID NOs: 30-35, and (b) a class 2, type V Cas endonuclease configured to bind to the engineered guide RNA. In some embodiments, the guide RNA comprises a sequence complementary to a eukaryotic, fungal, plant, mammalian, or human genomic polynucleotide sequence. In some embodiments, the guide RNA is 30-250 nucleotides in length. In some embodiments, the endonuclease comprises one or more nuclear localization sequences (NLSs) proximal to an N- or C-terminus of the endonuclease. In some embodiments, the NLS comprises a sequence at least 80% identical to a sequence from the group consisting of SEQ ID NO: 36-51.

In some embodiments, the engineered nuclease system further comprises a single- or double-stranded DNA repair template comprising from 5′ to 3′: a first homology arm comprising a sequence of at least 20 nucleotides 5′ to the target deoxyribonucleic acid sequence, a synthetic DNA sequence of at least 10 nucleotides, and a second homology arm comprising a sequence of at least 20 nucleotides 3′ to the target sequence. In some embodiments, the first or second homology arm comprises a sequence of at least 40, 80, 120, 150, 200, 300, 500, or 1,000 nucleotides. In some embodiments, the first and second homology arms are homologous to a genomic sequence of a prokaryote, bacteria, fungus, or eukaryote. In some embodiments, the single- or double-stranded DNA repair template comprises a transgene donor. In some embodiments, the engineered nuclease system further comprises a DNA repair template comprising a double-stranded DNA segment flanked by one or two single-stranded DNA segments. In some embodiments, the single-stranded DNA segments are conjugated to the 5′ ends of the double-stranded DNA segment. In some embodiments, the single stranded DNA segments are conjugated to the 3′ ends of the double-stranded DNA segment. In some embodiments, the single-stranded DNA segments have a length from 4 to 10 nucleotide bases. In some embodiments, the single-stranded DNA segments have a nucleotide sequence complementary to a sequence within the spacer sequence. In some embodiments, the double-stranded DNA sequence comprises a barcode, an open reading frame, an enhancer, a promoter, a protein-coding sequence, a miRNA coding sequence, an RNA coding sequence, or a transgene. In some embodiments, the double-stranded DNA sequence is flanked by a nuclease cut site. In some embodiments, the nuclease cut site comprises a spacer and a PAM sequence. In some embodiments, the system further comprises a source of Mg²⁺. In some embodiments, the guide RNA comprises a hairpin comprising at least 8, at least 10, or at least 12 base-paired ribonucleotides. In some embodiments, the hairpin comprises 10 base-paired ribonucleotides. In some embodiments, a) the endonuclease comprises a sequence at least 75%, 80%, or 90% identical to any one of SEQ ID NOs: 1-3, 11, 13, or 15, or a variant thereof; and b) the guide RNA structure comprises a sequence at least 80%, or 90% identical to the non-degenerate nucleotides of any one of SEQ ID NOs: 30-35. In some embodiments, the sequence identity is determined by a BLASTP, CLUSTALW, MUSCLE, MAFFT algorithm, or a CLUSTALW algorithm with the Smith-Waterman homology search algorithm parameters. In some embodiments, the sequence identity is determined by the BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

In some aspects, the present disclosure provides an engineered guide RNA comprising: a) a DNA-targeting segment comprising a nucleotide sequence that is complementary to a target sequence in a target DNA molecule; and b) a protein-binding segment comprising two complementary stretches of nucleotides that hybridize to form a double-stranded RNA (dsRNA) duplex, wherein the two complementary stretches of nucleotides are covalently linked to one another with intervening nucleotides, and wherein the engineered guide ribonucleic acid polynucleotide is capable of forming a complex with an endonuclease having at least 75% sequence identity to any one of SEQ ID NOs: 1-15, and targeting the complex to the target sequence of the target DNA molecule. In some embodiments, the DNA-targeting segment is positioned 3′ of both of the two complementary stretches of nucleotides. In some embodiments, the protein binding segment comprises a sequence having at least 70%, at least 80%, or at least 90% identity to the non-degenerate nucleotides of SEQ ID NO: 30-35. In some embodiments, the double-stranded RNA (dsRNA) duplex comprises at least 5, at least 8, at least 10, or at least 12 ribonucleotides.

In some aspects, the present disclosure provides a deoxyribonucleic acid polynucleotide encoding an engineered guide ribonucleic acid polynucleotide described herein.

In some aspects, the present disclosure provides a nucleic acid comprising an engineered nucleic acid sequence optimized for expression in an organism, wherein the nucleic acid encodes a class 2, type V Cas endonuclease, and wherein the endonuclease is derived from an uncultivated microorganism, wherein the organism is not the uncultivated organism. In some embodiments, the endonuclease comprises a variant having at least 70% or at least 80% sequence identity to any one of SEQ ID NOs: 1-15. In some embodiments, the endonuclease comprises a sequence encoding one or more nuclear localization sequences (NLSs) proximal to an N- or C-terminus of the endonuclease. In some embodiments, the NLS comprises a sequence selected from SEQ ID NOs: 36-51. In some embodiments, the NLS comprises SEQ ID NO: 37. In some embodiments, the NLS is proximal to the N-terminus of the endonuclease. In some embodiments, the NLS comprises SEQ ID NO: 36. In some embodiments, the NLS is proximal to the C-terminus of the endonuclease. In some embodiments, the organism is prokaryotic, bacterial, eukaryotic, fungal, plant, mammalian, rodent, or human.

In some aspects, the present disclosure provides an engineered vector comprising a nucleic acid sequence encoding a class 2, type V Cas endonuclease, wherein the endonuclease is derived from an uncultivated microorganism.

In some aspects, the present disclosure provides an engineered vector comprising a nucleic acid described herein.

In some aspects, the present disclosure provides an engineered vector comprising a deoxyribonucleic acid polynucleotide described herein. In some embodiments, the vector is a plasmid, a minicircle, a CELiD, an adeno-associated virus (AAV) derived virion, a lentivirus, or an adenovirus.

In some aspects, the present disclosure provides a cell comprising a vector described herein.

In some aspects, the present disclosure provides a method of manufacturing an endonuclease, comprising cultivating any of the host cells described herein.

In some aspects, the present disclosure provides a method for binding, cleaving, marking, or modifying a double-stranded deoxyribonucleic acid polynucleotide, comprising: (a) contacting the double-stranded deoxyribonucleic acid polynucleotide with a class 2, type V Cas endonuclease in complex with an engineered guide RNA configured to bind to the endonuclease and the double-stranded deoxyribonucleic acid polynucleotide: wherein the double-stranded deoxyribonucleic acid polynucleotide comprises a protospacer adjacent motif (PAM); and wherein the guide RNA structure comprises a sequence at least 80%, or 90% identical to the non-degenerate nucleotides of any one of SEQ ID NOs: 30-35. In some embodiments, the double-stranded deoxyribonucleic acid polynucleotide comprises a first strand comprising a sequence complementary to a sequence of the engineered guide RNA and a second strand comprising the PAM. In some embodiments, the PAM is directly adjacent to the 5′ end of the sequence complementary to the sequence of the engineered guide RNA. In some embodiments, the class 2, type V Cas endonuclease is derived from an uncultivated microorganism. In some embodiments, the double-stranded deoxyribonucleic acid polynucleotide is a eukaryotic, plant, fungal, mammalian, rodent, or human double-stranded deoxyribonucleic acid polynucleotide.

In some aspects, the present disclosure provides a method of modifying a target nucleic acid locus, the method comprising delivering to the target nucleic acid locus the engineered nuclease system described herein, wherein the endonuclease is configured to form a complex with the engineered guide ribonucleic acid structure, and wherein the complex is configured such that upon binding of the complex to the target nucleic acid locus, the complex modifies the target nucleic acid locus. In some embodiments, modifying the target nucleic acid locus comprises binding, nicking, cleaving, or marking the target nucleic acid locus. In some embodiments, the target nucleic acid locus comprises deoxyribonucleic acid (DNA) or ribonucleic acid (RNA). In some embodiments, the target nucleic acid comprises genomic DNA, viral DNA, viral RNA, or bacterial DNA. In some embodiments, the target nucleic acid locus is in vitro. In some embodiments, the target nucleic acid locus is within a cell. In some embodiments, the cell is a prokaryotic cell, a bacterial cell, a eukaryotic cell, a fungal cell, a plant cell, an animal cell, a mammalian cell, a rodent cell, a primate cell, a human cell, or a primary cell. In some embodiments, the cell is a primary cell. In some embodiments, the primary cell is a T cell. In some embodiments, the primary cell is a hematopoietic stem cell (HSC). In some embodiments, delivering the engineered nuclease system to the target nucleic acid locus comprises delivering a nucleic acid described herein or a vector described herein. In some embodiments, delivering the engineered nuclease system to the target nucleic acid locus comprises delivering a nucleic acid comprising an open reading frame encoding the endonuclease. In some embodiments, the nucleic acid comprises a promoter to which the open reading frame encoding the endonuclease is operably linked. In some embodiments, delivering the engineered nuclease system to the target nucleic acid locus comprises delivering a capped mRNA containing the open reading frame encoding the endonuclease. In some embodiments, delivering the engineered nuclease system to the target nucleic acid locus comprises delivering a translated polypeptide. In some embodiments, delivering the engineered nuclease system to the target nucleic acid locus comprises delivering a deoxyribonucleic acid (DNA) encoding the engineered guide RNA operably linked to a ribonucleic acid (RNA) pol III promoter. In some embodiments, the endonuclease induces a single-stranded break or a double-stranded break at or proximal to the target locus. In some embodiments, the endonuclease induces a staggered single stranded break within or 3′ to the target locus.

In some aspects, the present disclosure provides a host cell comprising an open reading frame encoding a heterologous endonuclease having at least 75% sequence identity to any one of SEQ ID NOs: 1-15 or a variant thereof. In some embodiments, the endonuclease has at least 75% sequence identity to any one of SEQ ID NOs: 1-3, 11, 13, or 15, or a variant thereof. In some embodiments, the host cell is an E, coli cell or a mammalian cell. In some embodiments, the host cell is an E, coli cell. In some embodiments, the E, coli cell is a λDE3 lysogen or the E, coli cell is a BL21 (DE3) strain. In some embodiments, the E, coli cell has an ompT lon genotype. In some embodiments, the open reading frame is operably linked to a T7 promoter sequence, a T7-lac promoter sequence, a lac promoter sequence, a tac promoter sequence, a trc promoter sequence, a ParaBAD promoter sequence, a PrhaBAD promoter sequence, a T5 promoter sequence, a cspA promoter sequence, an aralBAD promoter, a strong leftward promoter from phage lambda (pL promoter), or any combination thereof. In some embodiments, the open reading frame comprises a sequence encoding an affinity tag linked in-frame to a sequence encoding the endonuclease. In some embodiments, the affinity tag is an immobilized metal affinity chromatography (IMAC) tag. In some embodiments, the IMAC tag is a polyhistidine tag. In some embodiments, the affinity tag is a myc tag, a human influenza hemagglutinin (HA) tag, a maltose binding protein (MBP) tag, a glutathione S-transferase (GST) tag, a streptavidin tag, a FLAG tag, or any combination thereof. In some embodiments, the affinity tag is linked in-frame to the sequence encoding the endonuclease via a linker sequence encoding a protease cleavage site. In some embodiments, the protease cleavage site is a tobacco etch virus (TEV) protease cleavage site, a PreScission R: protease cleavage site, a Thrombin cleavage site, a Factor Xa cleavage site, an enterokinase cleavage site, or any combination thereof. In some embodiments, open reading frame is codon-optimized for expression in the host cell. In some embodiments, the open reading frame is provided on a vector. In some embodiments, the open reading frame is integrated into a genome of the host cell.

In some aspects, the present disclosure provides a culture comprising any of the host cells described herein in compatible liquid medium.

In some aspects, the present disclosure provides a method of producing an endonuclease, comprising cultivating any of the host cells described herein in compatible growth medium. In some embodiments, the method further comprises inducing expression of the endonuclease by addition of an additional chemical agent or an increased amount of a nutrient. In some embodiments, an additional chemical agent or an increased amount of a nutrient comprises Isopropyl β-D-1-thiogalactopyranoside (IPTG) or additional amounts of lactose. In some embodiments, the method further comprises isolating the host cell after the cultivation and lysing the host cell to produce a protein extract. In some embodiments, the method further comprises subjecting the protein extract to IMAC, or ion-affinity chromatography. In some embodiments, the open reading frame comprises a sequence encoding an IMAC affinity tag linked in-frame to a sequence encoding the endonuclease. In some embodiments, the IMAC affinity tag is linked in-frame to the sequence encoding the endonuclease via a linker sequence encoding protease cleavage site. In some embodiments, the protease cleavage site comprises a tobacco etch virus (TEV) protease cleavage site, a PreScissionR; protease cleavage site, a Thrombin cleavage site, a Factor Xa cleavage site, an enterokinase cleavage site, or any combination thereof. In some embodiments, the method further comprises cleaving the IMAC affinity tag by contacting a protease corresponding to the protease cleavage site to the endonuclease. In some embodiments, the method further comprises performing subtractive IMAC affinity chromatography to remove the affinity tag from a composition comprising the endonuclease.

In some aspects, the present disclosure provides a method of disrupting a locus in a cell, comprising contacting to the cell a composition comprising: (a) a class 2, type V Cas endonuclease having at least 75% identity to any one of SEQ ID NOs: 1-15 or a variant thereof; and (b) an engineered guide RNA, wherein the engineered guide RNA is configured to form a complex with the endonuclease and the engineered guide RNA comprises a spacer sequence configured to hybridize to a region of the locus, wherein the class 2, type V Cas endonuclease has at least equivalent cleavage activity to spCas9 in the cell. In some embodiments, the cleavage activity is measured in vitro by introducing the endonucleases alongside compatible guide RNAs to cells comprising the target nucleic acid and detecting cleavage of the target nucleic acid sequence in the cells. In some embodiments, the composition comprises 20 pmoles or less of the class 2, type V Cas endonuclease. In some embodiments, the composition comprises 1 pmol or less of the class 2, type V Cas endonuclease.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts organizations of CRISPR/Cas loci of different classes and types.

FIG. 2A-2C depicts the MG119 Family. FIG. 2A depicts a multiple alignment of MG119 effectors representatives showing domains compositions and conservation of the RuvC catalytic residues critical for function for a double stranded DNA cleavage activity. FIG. 2B depicts a representation of a CRISPR-containing contig with genomic context surrounding the CRISPR array and the Cas effector (example of MG119-60). FIG. 2C depicts folding of the Direct repeat of MG119-60. Figure discloses SEQ ID NO: 52.

FIG. 3A-3D depicts the MG90 Family. FIG. 3A depicts a multiple alignment of MG90 effectors representatives showing domains compositions and conservation of the RuvC catalytic residues critical for function for a double stranded DNA cleavage activity. FIG. 3B depicts a representation of a CRISPR-containing contig with genomic context surrounding the CRISPR array and the Cas effector (example of MG90-4). FIG. 3C depicts folding of the Direct repeat of MG90-4. Figure discloses SEQ ID NO: 53. FIG. 3D depicts a single guide RNA designed for MG90-4. Figure discloses SEQ ID NO: 33.

FIG. 4A-4C depicts the MG127 Family. FIG. 4A depicts a multiple alignment of MG127 effectors representatives showing domains compositions and conservation of the RuvC catalytic residues critical for function for a double stranded DNA cleavage activity. FIG. 4B depicts a representation of a CRISPR-containing contig with genomic context surrounding the CRISPR array and the Cas effector (example of MG127-1). FIG. 4C depicts folding of the Direct repeat of MG127-1. Figure discloses SEQ ID NO: 54.

FIG. 5A-5C depicts the MG126 Family. FIG. 5A depicts a multiple alignment of MG126 effectors representatives showing domains compositions and conservation of the RuvC catalytic residues critical for function for a double stranded DNA cleavage activity. FIG. 5B depicts a representation of a CRISPR-containing contig with genomic context surrounding the CRISPR array and the Cas effector (example of MG126-1). FIG. 5C depicts folding of the Direct repeat of MG126-1. Figure discloses SEQ ID NO: 55.

FIG. 6A-6C depicts the MG118 Family. FIG. 6A depicts a multiple alignment of MG118 effectors representatives showing domains compositions and conservation of the RuvC catalytic residues critical for function for a double stranded DNA cleavage activity. FIG. 6B depicts a representation of a CRISPR-containing contig with genomic context surrounding the CRISPR array and the Cas effector (example of MG118-2). FIG. 6C depicts folding of the Direct repeat of MG118-2. Figure discloses SEQ ID NO: 56.

FIG. 7A-7C depicts the MG120 Family. FIG. 7A depicts a multiple alignment of MG120 effectors representatives showing domains compositions and conservation of the RuvC catalytic residues critical for function for a double stranded DNA cleavage activity. FIG. 7B depicts a representation of a CRISPR-containing contig with genomic context surrounding the CRISPR array and the Cas effector (example of MG120-10). FIG. 7C depicts folding of the Direct repeat of MG120-10. Figure discloses SEQ ID NO: 57.

### BRIEF DESCRIPTION OF THE SEQUENCE LISTING

The Sequence Listing filed herewith provides exemplary polynucleotide and polypeptide sequences for use in methods, compositions, and systems according to the disclosure. Below are exemplary descriptions of sequences therein.

### MG120

SEQ ID NO: 1 shows the full-length peptide sequence of a MG120 nuclease.

### MG118

SEQ ID NO: 2 shows the full-length peptide sequence of an MG118 nuclease.

SEQ ID NOs: 22-23 show nucleotide sequences of MG118 minimal arrays.

SEQ ID NOs: 28-29 show nucleotide sequences of MG118 target CRISPR repeats.

SEQ ID NOs: 30-31 show nucleotide sequences of MG118 crRNAs.

### MG90

SEQ ID NOs: 3-10 show the full-length peptide sequences of MG90 nucleases.

SEQ ID NOs: 16-21 show nucleotide sequences of MG90 tracrRNAs derived from the same loci as a MG90) Cas effector.

SEQ ID NOs: 24-27 show nucleotide sequences of MG90 minimal arrays.

SEQ ID NOs: 32-33 show nucleotide sequences of MG90 target CRISPR repeats.

SEQ ID NOs: 34-35 show nucleotide sequences of MG90 sgRNAs.

### MG119

SEQ ID NOs: 11-12 show the full-length peptide sequences of MG119 nucleases.

### MG126

SEQ ID NOs: 13-14 show the full-length peptide sequences of MG126 nucleases.

### MG127

SEQ ID NO: 15 shows the full-length peptide sequence of a MG127 nuclease.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The practice of some methods disclosed herein employ, unless otherwise indicated, techniques of immunology, biochemistry, chemistry, molecular biology, microbiology, cell biology, genomics, and recombinant DNA. See for example Sambrook and Green. Molecular Cloning: A Laboratory Manual. 4th Edition (2012): the series Current Protocols in Molecular Biology (F. M. Ausubel, et al, eds.); the series Methods In Enzymology (Academic Press, Inc.). PCR 2: A Practical Approach (M. J. MacPherson. B. D. Hames and G. R. Taylor eds. (1995)). Harlow and Lane, eds. (1988) Antibodies. A Laboratory Manual, and Culture of Animal Cells: A Manual of Basic Technique and Specialized Applications. 6th Edition (R. I. Freshney, ed. (2010))) (which is entirely incorporated by reference herein).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined. i.e., the limitations of the measurement system. For example. “about” can mean within one or more than one standard deviation, per the practice in the art. Alternatively, “about” can mean a range of up to 20%, up to 15%, up to 10%, up to 5%, or up to 1% of a given value.

As used herein, a “cell” generally refers to a biological cell. A cell may be the basic structural, functional and/or biological unit of a living organism. A cell may originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton. cannabis, tobacco, flowering plants, conifers, gymnosperms, ferns, clubmosses, hornworts, liverworts, mosses), an algal cell. (e.g., Botryococcus braunii. Chlamydomonas reinhardtii. Nannochloropsis gaditana. Chlorella pyrenoidosa, Sargassum patens C. agardh, and the like), seaweeds (e.g., kelp), a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

The term “nucleotide.” as used herein, generally refers to a base-sugar-phosphate combination. A nucleotide may comprise a synthetic nucleotide. A nucleotide may comprise a synthetic nucleotide analog. Nucleotides may be monomeric units of a nucleic acid sequence (e.g., deoxyribonucleic acid (DNA) and ribonucleic acid (RNA)). The term nucleotide may include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives may include, for example, [αS]dATP, 7-deaza-dGTP and 7-deaza-dATP, and nucleotide derivatives that confer nuclease resistance on the nucleic acid molecule containing them. The term nucleotide as used herein may refer to dideoxyribonucleoside triphosphates (ddNTPs) and their derivatives. Illustrative examples of dideoxyribonucleoside triphosphates may include, but are not limited to, ddATP, ddCTP, ddGTP, ddITP, and ddTTP. A nucleotide may be unlabeled or detectably labeled, such as using moieties comprising optically detectable moieties (e.g., fluorophores). Labeling may also be carried out with quantum dots. Detectable labels may include, for example, radioactive isotopes, fluorescent labels, chemiluminescent labels, bioluminescent labels, and enzyme labels. Fluorescent labels of nucleotides may include but are not limited fluorescein. 5-carboxyfluorescein (FAM). 2′7′-dimethoxy-4′5-dichloro-6-carboxyfluorescein (JOE), rhodamine. 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green. Texas Red. Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP. [ROX]ddTTP, [dR6G]ddATP, [dR110]ddCTP, [dTAMRA]ddGTP, and [dROX]ddTTP available from Perkin Elmer. Foster City. Calif: FluoroLink Deoxy Nucleotides, FluoroLink Cy3-dCTP. FluoroLink Cy5-dCTP. FluoroLink Fluor X-dCTP. FluoroLink Cy3-dUTP, and FluoroLink Cy5-dUTP available from Amersham. Arlington Heights. Il.: Fluorescein-15-dATP. Fluorescein-12-dUTP. Tetramethyl-rodamine-6-dUTP. IR 770-9-dATP. Fluorescein-12-ddUTP, Fluorescein-12-UTP, and Fluorescein-15-2′-dATP available from Boehringer Mannheim. Indianapolis. Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g., biotin-11-dUTP, biotin-16-dUTP, biotin-20-dUTP).

The terms “polynucleotide.” “oligonucleotide.” and “nucleic acid” are used interchangeably to generally refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure and may perform any function. A polynucleotide may comprise one or more analogs (e.g., altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin. 7-deaza-GTP, fluorophores (e.g., rhodamine or fluorescein linked to the sugar), thiol-containing nucleotides, biotin-linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

The terms “transfection” or “transfected” generally refer to introduction of a nucleic acid into a cell by non-viral or viral-based methods. The nucleic acid molecules may be gene sequences encoding complete proteins or functional portions thereof. See, e.g., Sambrook et al. 1989, Molecular Cloning: A Laboratory Manual, 18.1-18.88 (which is entirely incorporated by reference herein).

The terms “peptide.” “polypeptide.” and “protein” are used interchangeably herein to generally refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer may be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids.” as used herein, generally refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

As used herein, the “non-native” can generally refer to a nucleic acid or polypeptide sequence that is not found in a native nucleic acid or protein. Non-native may refer to affinity tags. Non-native may refer to fusions. Non-native may refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that may also be exhibited by the nucleic acid and/or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide.

The term “promoter”, as used herein, generally refers to the regulatory DNA region which controls transcription or expression of a gene, and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. A ‘basal promoter’, also referred to as a core promoter, may generally refer to a promoter that contains all the basic necessary elements to promote transcriptional expression of an operably linked polynucleotide. Eukaryotic basal promoters typically, though not necessarily, contain a TATA-box and/or a CAAT box.

The term “expression”, as used herein, generally refers to the process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “operably linked”, “operable linkage”, “operatively linked”, or grammatical equivalents thereof generally refer to juxtaposition of genetic elements, e.g., a promoter, an enhancer, a polyadenylation sequence, etc., wherein the elements are in a relationship permitting them to operate in the expected manner. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “vector” as used herein, generally refers to a macromolecule or association of macromolecules that comprises or associates with a polynucleotide and which may be used to mediate delivery of the polynucleotide to a cell. Examples of vectors include plasmids, viral vectors, liposomes, and other gene delivery vehicles. The vector generally comprises genetic elements, e.g., regulatory elements, operatively linked to a gene to facilitate expression of the gene in a target.

As used herein. “an expression cassette” and “a nucleic acid cassette” are used interchangeably generally to refer to a combination of nucleic acid sequences or elements that are expressed together or are operably linked for expression. In some cases, an expression cassette refers to the combination of regulatory elements and a gene or genes to which they are operably linked for expression.

A “functional fragment” of a DNA or protein sequence generally refers to a fragment that retains a biological activity (either functional or structural) that is substantially similar to a biological activity of the full-length DNA or protein sequence. A biological activity of a DNA sequence may be its ability to influence expression in a manner known to be attributed to the full-length sequence.

As used herein, an “engineered” object generally indicates that the object has been modified by human intervention. According to non-limiting examples: a nucleic acid may be modified by changing its sequence to a sequence that does not occur in nature; a nucleic acid may be modified by ligating it to a nucleic acid that it does not associate with in nature such that the ligated product possesses a function not present in the original nucleic acid; an engineered nucleic acid may synthesized in vitro with a sequence that does not exist in nature; a protein may be modified by changing its amino acid sequence to a sequence that does not exist in nature; an engineered protein may acquire a new function or property. An “engineered” system comprises at least one engineered component.

As used herein. “synthetic” and “artificial” can generally be used interchangeably to refer to a protein or a domain thereof that has low sequence identity (e.g., less than 50% sequence identity, less than 25% sequence identity, less than 10% sequence identity, less than 5% sequence identity, less than 1% sequence identity) to a naturally occurring human protein. For example, VPR and VP64 domains are synthetic transactivation domains.

As used herein, the term “Cas12a” generally refers to a family of Cas endonucleases that are class 2. Type V-A Cas endonucleases and that (a) use a relatively small guide RNA (about 42-44 nucleotides) that is processed by the nuclease itself following transcription from the CRISPR array, and (b) cleave DNA to leave staggered cut sites. Further features of this family of enzymes can be found, e.g, in Zetsche B. Heidenreich M. Mohanraju P, et al. Nat Biotechnol 2017; 35:31-34, and Zetsche B. Gootenberg J S. Abudayyeh O O, et al. Cell 2015; 163:759-771, which are incorporated by reference herein.

As used herein, a “guide nucleic acid” can generally refer to a nucleic acid that may hybridize to another nucleic acid. A guide nucleic acid may be RNA. A guide nucleic acid may be DNA. The guide nucleic acid may be programmed to bind to a sequence of nucleic acid site-specifically. The nucleic acid to be targeted, or the target nucleic acid, may comprise nucleotides. The guide nucleic acid may comprise nucleotides. A portion of the target nucleic acid may be complementary to a portion of the guide nucleic acid. The strand of a double-stranded target polynucleotide that is complementary to and hybridizes with the guide nucleic acid may be called the complementary strand. The strand of the double-stranded target polynucleotide that is complementary to the complementary strand, and therefore may not be complementary to the guide nucleic acid may be called noncomplementary strand. A guide nucleic acid may comprise a polynucleotide chain and can be called a “single guide nucleic acid.” A guide nucleic acid may comprise two polynucleotide chains and may be called a “double guide nucleic acid.” If not otherwise specified, the term “guide nucleic acid” may be inclusive, referring to both single guide nucleic acids and double guide nucleic acids. A guide nucleic acid may comprise a segment that can be referred to as a “nucleic acid-targeting segment” or a “nucleic acid-targeting sequence” or “spacer sequence.” A nucleic acid-targeting segment may comprise a sub-segment that may be referred to as a “protein binding segment” or “protein binding sequence” or “Cas protein binding segment”.

The term “sequence identity” or “percent identity” in the context of two or more nucleic acids or polypeptide sequences, generally refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues: BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov): CLUSTALW with the Smith-Waterman homology search algorithm parameters with a match of 2, a mismatch of −1, and a gap of −1; MUSCLE with default parameters: MAFFT with parameters of a retree of 2 and max iterations of 1000; Novafold with default parameters: HMMER hmmalign with default parameters.

The term “optimally aligned” in the context of two or more nucleic acids or polypeptide sequences, generally refers to two (e.g., in a pairwise alignment) or more (e.g., in a multiple sequence alignment) sequences that have been aligned to maximal correspondence of amino acids residues or nucleotides, for example, as determined by the alignment producing a highest or “optimized” percent identity score.

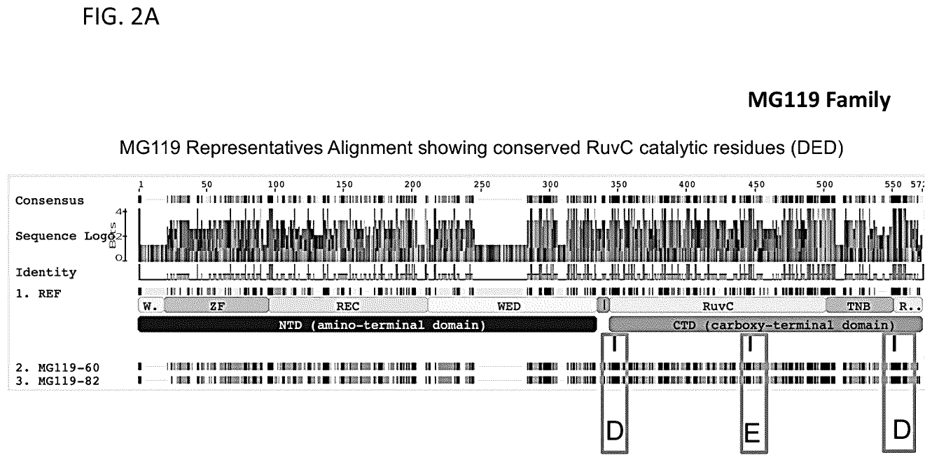

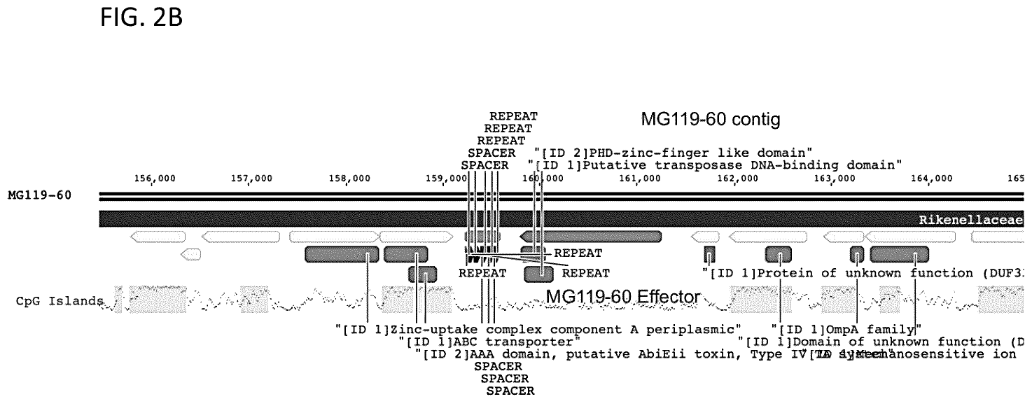

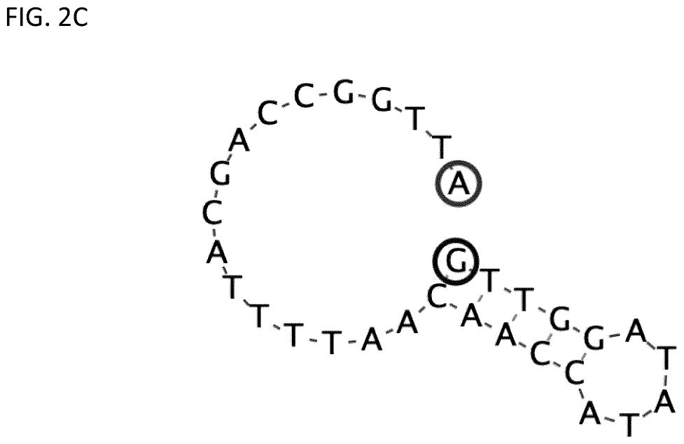

Included in the current disclosure are variants of any of the enzymes described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally, or alternatively, by comparing aligned sequences of homologous proteins from different species, conservative substitutions can be identified by locating amino acid residues that have been mutated between species (e.g., non-conserved residues) without altering the basic functions of the encoded proteins. Such conservatively substituted variants may include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% identity to any one of the endonuclease protein sequences described herein (e.g. MG90, MG118, MG119, MG120, MG126, or MG127 family endonucleases described herein, or any other family nuclease described herein). In some embodiments, such conservatively substituted variants are functional variants. Such functional variants can encompass sequences with substitutions such that the activity of one or more critical active site residues or guide RNA binding residues of the endonuclease are not disrupted. In some embodiments, a functional variant of any of the proteins described herein lacks substitution of at least one of the conserved or functional residues called out in FIG. 2A, 3A, 4A, 5A, or 6A. In some embodiments, a functional variant of any of the proteins described herein lacks substitution of all of the conserved or functional residues called out in FIG. 2A, 3A, 4A, 5A, or 6A.

Also included in the current disclosure are variants of any of the enzymes described herein with substitution of one or more catalytic residues to decrease or eliminate activity of the enzyme (e.g. decreased-activity variants). In some embodiments, a decreased activity variant as a protein described herein comprises a disrupting substitution of at least one, at least two, or all three catalytic residues called out in FIG. 2A, 3A, 4A, 5A, or 6A.

Conservative substitution tables providing functionally similar amino acids are available from a variety of references (see, for e.g., Creighton, Proteins: Structures and Molecular Properties (W H Freeman & Co.: 2nd edition (December 1993)). The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)

### Overview

The discovery of new Cas enzymes with unique functionality and structure may offer the potential to further disrupt deoxyribonucleic acid (DNA) editing technologies, improving speed, specificity, functionality, and ease of use. Relative to the predicted prevalence of Clustered Regularly Interspaced Short Palindromic Repeats (CRISPR) systems in microbes and the sheer diversity of microbial species, relatively few functionally characterized CRISPR/Cas enzymes exist in the literature. This is partly because a huge number of microbial species may not be readily cultivated in laboratory conditions. Metagenomic sequencing from natural environmental niches containing large numbers of microbial species may offer the potential to drastically increase the number of new CRISPR/Cas systems known and speed the discovery of new oligonucleotide editing functionalities. A recent example of the fruitfulness of such an approach is demonstrated by the 2016 discovery of CasX/CasY CRISPR systems from metagenomic analysis of natural microbial communities.

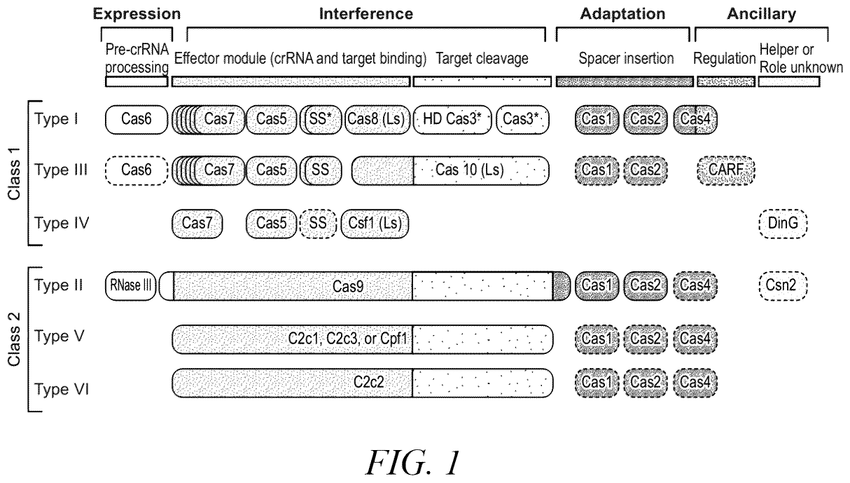

CRISPR/Cas systems are RNA-directed nuclease complexes that have been described to function as an adaptive immune system in microbes. In their natural context, CRISPR/Cas systems occur in CRISPR (clustered regularly interspaced short palindromic repeats) operons or loci, which generally comprise two parts: (i) an array of short repetitive sequences (30-40 bp) separated by equally short spacer sequences, which encode the RNA-based targeting element; and (ii) ORFs encoding the Cas encoding the nuclease polypeptide directed by the RNA-based targeting element alongside accessory proteins/enzymes. Efficient nuclease targeting of a particular target nucleic acid sequence generally requires both (i) complementary hybridization between the first 6-8 nucleic acids of the target (the target seed) and the crRNA guide; and (ii) the presence of a protospacer-adjacent motif (PAM) sequence within a defined vicinity of the target seed (the PAM usually being a sequence not commonly represented within the host genome). Depending on the exact function and organization of the system, CRISPR-Cas systems are commonly organized into 2 classes, 5 types, and 16 subtypes based on shared functional characteristics and evolutionary similarity (see FIG. 1).

Class I CRISPR-Cas systems have large, multi-subunit effector complexes, and comprise Types I, III, and IV. Class II CRISPR-Cas systems generally have single-polypeptide multidomain nuclease effectors, and comprise Types II, V and VI.

Type II CRISPR-Cas systems are considered the simplest in terms of components. In Type II CRISPR-Cas systems, the processing of the CRISPR array into mature crRNAs does not require the presence of a special endonuclease subunit, but rather a small trans-encoded crRNA (tracrRNA) with a region complementary to the array repeat sequence: the tracrRNA interacts with both its corresponding effector nuclease (e.g. Cas9) and the repeat sequence to form a precursor dsRNA structure, which is cleaved by endogenous RNAse III to generate a mature effector enzyme loaded with both tracrRNA and crRNA. Cas II nucleases are known as DNA nucleases. Type 2 effectors generally exhibit a structure consisting of a RuvC-like endonuclease domain that adopts the RNase H fold with an unrelated HNH nuclease domain inserted within the folds of the RuvC-like nuclease domain. The RuvC-like domain is responsible for the cleavage of the target (e.g., crRNA complementary) DNA strand, while the HNH domain is responsible for cleavage of the displaced DNA strand.

Type V CRISPR-Cas systems are characterized by a nuclease effector (e.g. Cas12) structure similar to that of Type II effectors, comprising a RuvC-like domain. Similar to Type II, most (but not all) Type V CRISPR systems use a tracrRNA to process pre-crRNAs into mature crRNAs; however, unlike Type II systems which requires RNAse III to cleave the pre-crRNA into multiple crRNAs, type V systems are capable of using the effector nuclease itself to cleave pre-crRNAs. Like Type-II CRISPR-Cas systems. Type V CRISPR-Cas systems are again known as DNA nucleases. Unlike Type II CRISPR-Cas systems, some Type V enzymes (e.g., Cas12a) appear to have a robust single-stranded nonspecific deoxyribonuclease activity that is activated by the first crRNA directed cleavage of a double-stranded target sequence.

CRISPR-Cas systems have emerged in recent years as the gene editing technology of choice due to their targetability and ease of use. The most commonly used systems are the Class 2 Type II SpCas9 and the Class 2 Type V-A Cas12a (previously Cpf1). The Type V-A systems in particular are becoming more widely used since their reported specificity in cells is higher than other nucleases, with fewer or no off-target effects. The V-A systems are also advantageous in that the guide RNA is small (42-44 nucleotides compared with approximately 100 nt for SpCas9) and is processed by the nuclease itself following transcription from the CRISPR array, simplifying multiplexed applications with multiple gene edits. Furthermore, the V-A systems have staggered cut sites, which may facilitate directed repair pathways, such as microhomology-dependent targeted integration (MITI).

The most commonly used Type V-A enzymes require a 5′ protospacer adjacent motif (PAM) next to the chosen target site: 5′-TTTV-3′ for Lachnospiraceae bacterium ND2006 LbCas12a and Acidaminococcus sp. AsCas12a; and 5′-TTV-3′ for Francisella novicida FnCas12a. Recent exploration of orthologs has revealed proteins with less restrictive PAM sequences that are also active in mammalian cell culture, for example YTV, YYN or TTN. However, these enzymes do not fully encompass Type V biodiversity and targetability, and may not represent all possible activities and PAM sequence requirements. Here, thousands of genomic fragments were mined from numerous metagenomes for Type V nucleases. The known diversity of V enzymes may have been expanded and novel systems may have been developed into highly targetable, compact, and precise gene editing agents.

### MG Enzymes

Type V CRISPR systems are quickly being adopted for use in a variety of genome editing applications. These programmable nucleases are part of adaptive microbial immune systems, the natural diversity of which has been largely unexplored. Novel families of Type V CRISPR enzymes were identified through a large-scale analysis of metagenomes collected from a variety of complex environments, and representatives of these were developed systems into gene-editing platforms. The majority of these systems come from uncultivated organisms, some of which encode a divergent Type V effector within the same CRISPR operon.

In some aspects, the present disclosure provides for novel Type V candidates. These candidates may represent one or more novel subtypes and some sub-families may have been identified. These nucleases are less than about 900 amino acids in length. These novel subtypes may be found in the same CRISPR locus as known Type V effectors. RuvC catalytic residues may have been identified for the novel Type V candidates, and these novel Type V candidates may not require tracrRNA.

In some aspects, the present disclosure provides for smaller Type V effectors. Such effectors may be small putative effectors. These effectors may simplify delivery and may extend therapeutic applications.

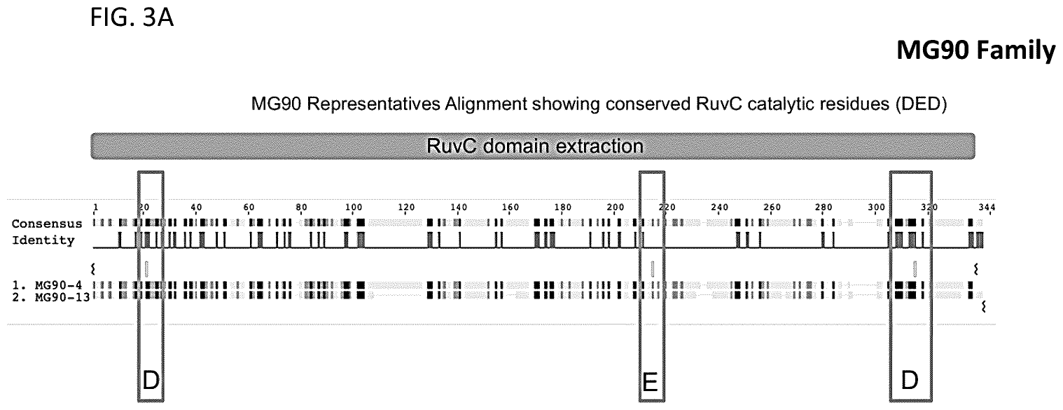

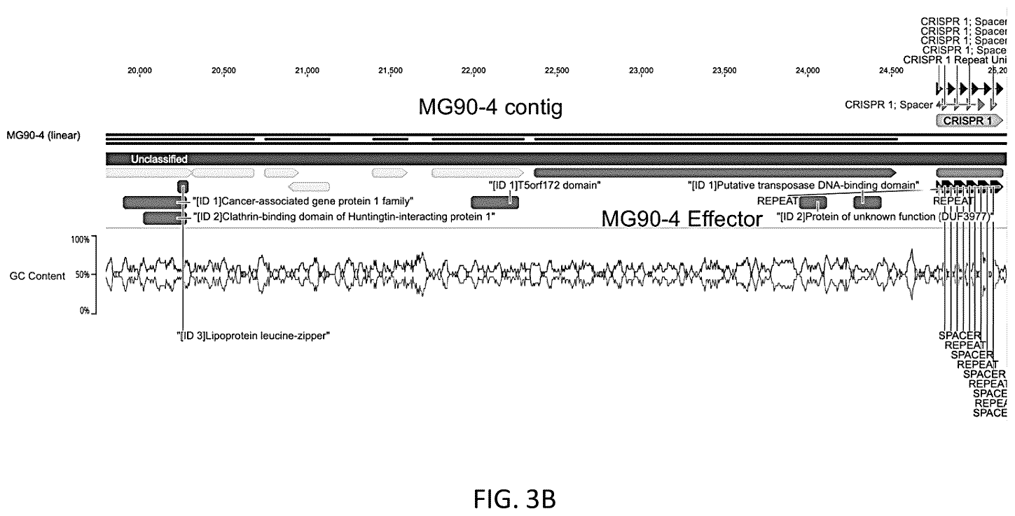

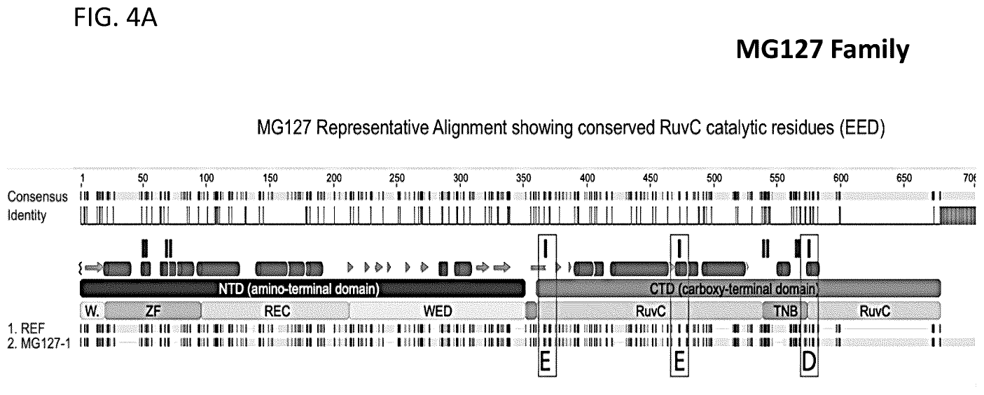

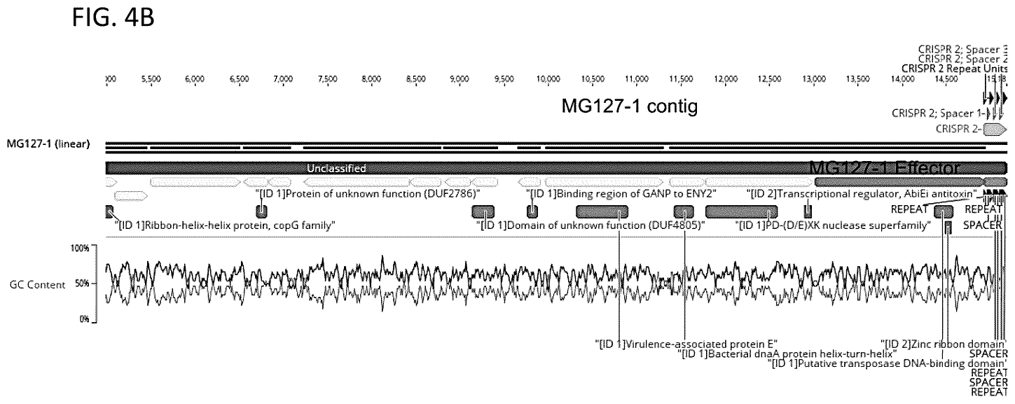

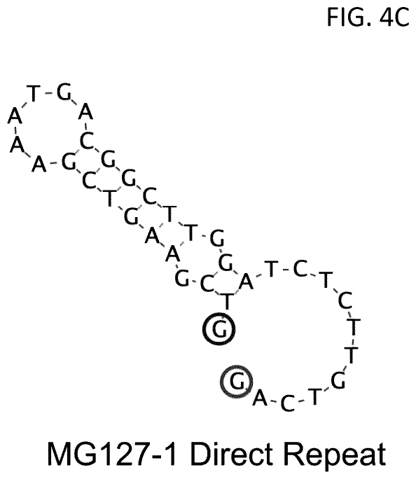

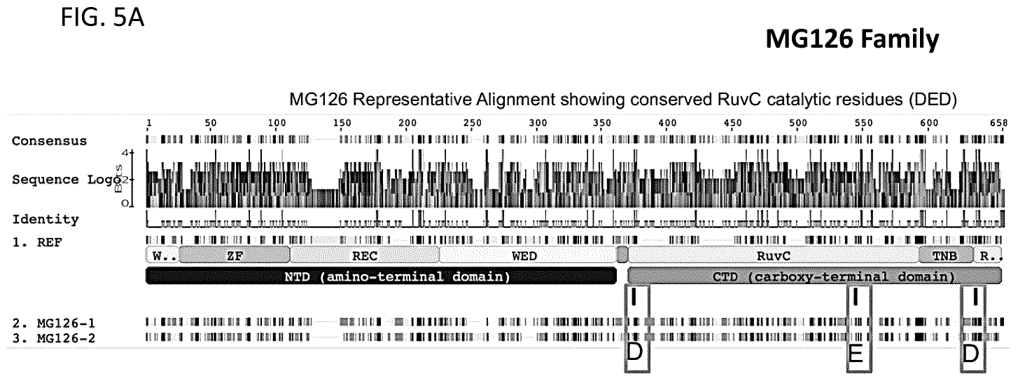

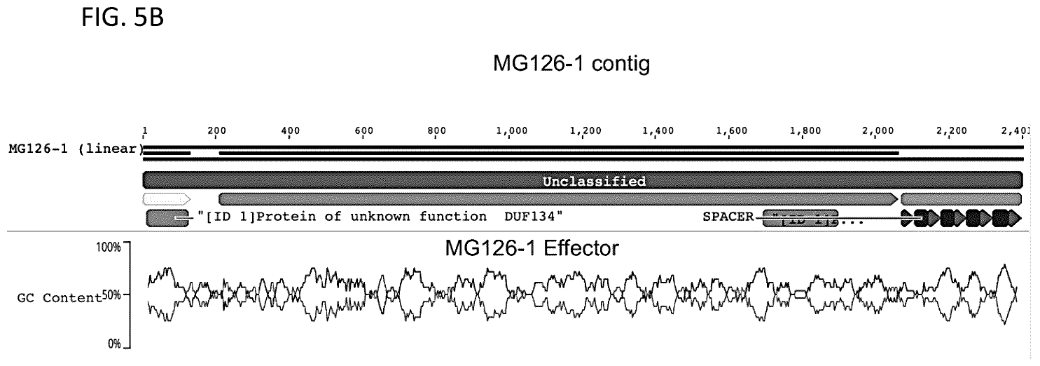

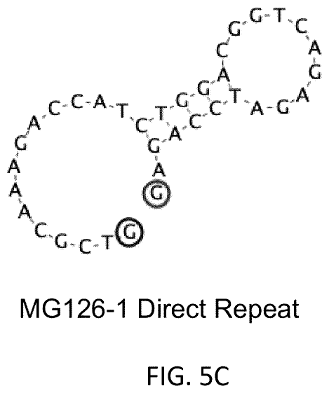

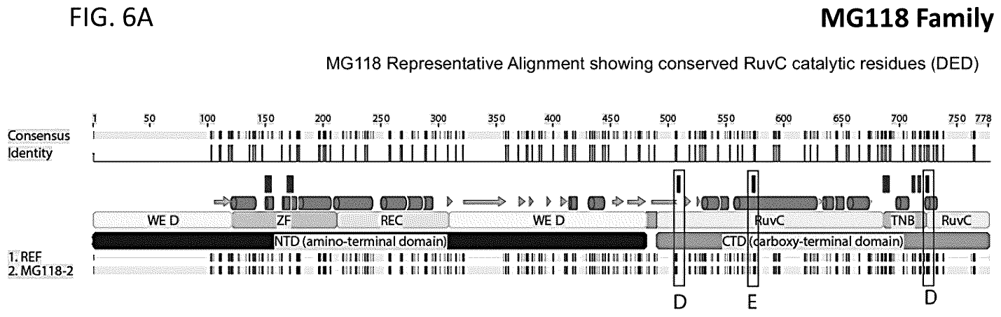

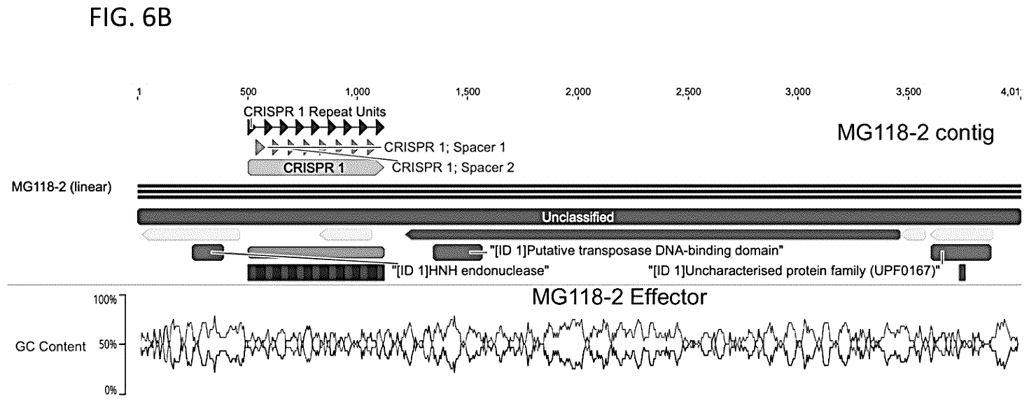

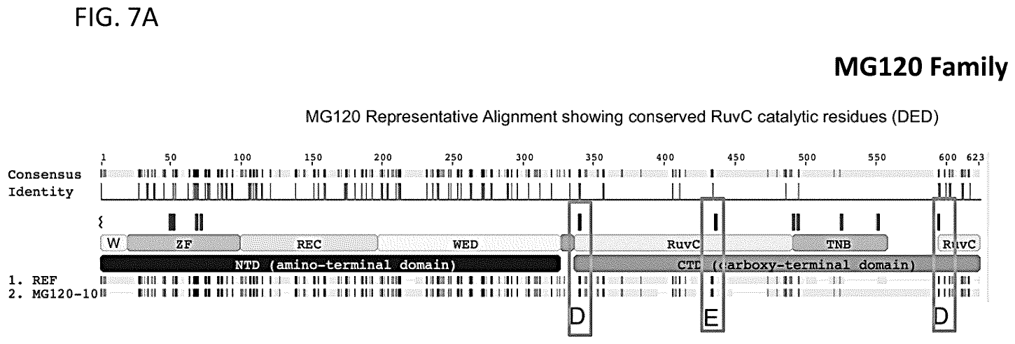

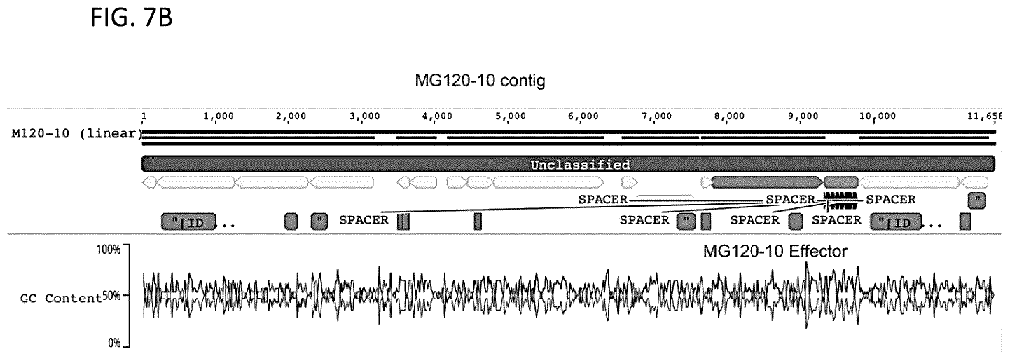

In some aspects, the present disclosure provides for a novel type V effector. Such an effector may be MG90 as described herein (see FIG. 3). Such an effector may be MG118 as described herein (see FIG. 6). Such an effector may be MG119 as described herein (see FIG. 2). Such an effector may be MG120) as described herein. Such an effector may be MG126 as described herein (see FIG. 5). Such an effector may be MG127 as described herein (see FIG. 4).

In one aspect, the present disclosure provides for an engineered nuclease system discovered through metagenomic sequencing. In some cases, the metagenomic sequencing is conducted on samples. In some cases, the samples may be collected from a variety of environments. Such environments may be a human microbiome, an animal microbiome, environments with high temperatures, environments with low temperatures. Such environments may include sediment.

In one aspect, the present disclosure provides for an engineered nuclease system comprising an endonuclease. In some cases, the endonuclease is a Cas endonuclease. In some cases, the endonuclease is a class 2, type V Cas endonuclease. In some cases, the endonuclease is a class 2, type V Cas endonuclease of a novel sub-type. In some cases, the endonuclease is derived from an uncultivated microorganism. The endonuclease may comprise a RuvC domain. In some cases, the engineered nuclease system comprises an engineered guide RNA. In some cases, the engineered guide RNA is configured to form a complex with the endonuclease. In some cases, the engineered guide RNA comprises a spacer sequence. In some cases, the spacer sequence is configured to hybridize to a target nucleic acid sequence.

In one aspect, the present disclosure provides for an engineered nuclease system comprising an endonuclease. In some cases, the endonuclease has at least about 70% sequence identity to any one of SEQ ID NOs: 1-15. In some cases, the endonuclease has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-15.

In some cases, the endonuclease comprises a variant having at least about 20%, at least about 25%, at least about 30%, at least about 35%, at least about 40%, at least about 45%, at least about 50) %, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-15. In some cases, the endonuclease may be substantially identical to any one of SEQ ID NOs: 1-15.

In some cases, the engineered nuclease system comprises an engineered guide RNA. In some cases, the engineered guide RNA is configured to form a complex with the endonuclease. In some cases, the engineered guide RNA comprises a spacer sequence. In some cases, the spacer sequence is configured to hybridize to a target nucleic acid sequence. In some cases, the endonuclease is configured to bind to a protospacer adjacent motif (PAM) sequence.

In some cases, the endonuclease is not a Cpf1 or Cms1 endonuclease.

In some cases, the guide RNA comprises a sequence with at least 80% sequence identity to the first 19 nucleotides or the non-degenerate nucleotides of SEQ ID NO: 30-35. In some cases, the guide RNA comprises a sequence with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80) %, at least about 85%, at least about 90%, at least about 91%, at least about 92%, at least about 93%, at least about 94%, at least about 95%, at least about 96%, at least about 97%, at least about 98%, or at least about 99% identity to the first 19 nucleotides or the non-degenerate nucleotides of SEQ ID NO: 30-35. In some cases, the guide RNA comprises a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to the first 19 nucleotides or the non-degenerate nucleotides of SEQ ID NO: 30-35. In some cases, the guide RNA comprises a sequence which is substantially identical to the first 19 nucleotides or the non-degenerate nucleotides of SEQ ID NO: 30-35.

In some cases, the guide RNA comprises a sequence with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to the first 19 nucleotides or the non-degenerate nucleotides of SEQ ID NO: 30-35. In some cases, the endonuclease is configured to bind to the engineered guide RNA. In some cases, the Cas endonuclease is configured to bind to the engineered guide RNA. In some cases, the class 2 Cas endonuclease is configured to bind to the engineered guide RNA. In some cases, the class 2, type V Cas endonuclease is configured to bind to the engineered guide RNA. In some cases, the class 2, type V, novel subtype Cas endonuclease is configured to bind to the engineered guide RNA.

In some cases, the guide RNA comprises a sequence complementary to a eukaryotic, fungal, plant, mammalian, or human genomic polynucleotide sequence. In some cases, the guide RNA comprises a sequence complementary to a eukaryotic genomic polynucleotide sequence. In some cases, the guide RNA comprises a sequence complementary to a fungal genomic polynucleotide sequence. In some cases, the guide RNA comprises a sequence complementary to a plant genomic polynucleotide sequence. In some cases, the guide RNA comprises a sequence complementary to a mammalian genomic polynucleotide sequence. In some cases, the guide RNA comprises a sequence complementary to a human genomic polynucleotide sequence.

In some cases, the guide RNA is 30-250 nucleotides in length. In some cases, the guide RNA is 42-44 nucleotides in length. In some cases, the guide RNA is 42 nucleotides in length. In some cases, the guide RNA is 43 nucleotides in length. In some cases, the guide RNA is 44 nucleotides in length. In some cases, the guide RNA is 85-245 nucleotides in length. In some cases, the guide RNA is more than 90 nucleotides in length. In some cases, the guide RNA is less than 245 nucleotides in length.

In some cases, the endonuclease may comprise a variant having one or more nuclear localization sequences (NLSs). The NLS may be proximal to the N- or C-terminus of the endonuclease. The NLS may be appended N-terminal or C-terminal to any one of SEQ ID NOs: 36-51, or to a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 36-51. In some cases, the NLS may comprise a sequence substantially identical to any one of SEQ ID NOs: 36-51.

TABLE 1
Example NLS Sequences that may be used with Cas Effectors according to the
disclosure.

| Source | NLS amino acid sequence | SEQ ID NO: |
|---|---|---|
| SV40 | PKKKRKV | 36 |
| nucleoplasmin | KRPAATKKAGQAKKKK | 37 |
| bipartite NLS | | |
| c-myc NLS | PAAKRVKLD | 38 |
| c-myc NLS | RQRRNELKRSP | 39 |
| hRNPA1 M9 NLS | NQSSNFGPMKGGNFGGRSSGPYGGGGQYFAKPRNQGGY | 40 |
| Importin-alpha IBB | RMRIZFKNKGKDTAELRRRRVEVSVELRKAKKDEQILKRRNV | 41 |
| domain | | |
| Myoma T protein | VSRKRPRP | 42 |
| Myoma T protein | PPKKARED | 43 |
| p53 | PQPKKKPL | 44 |
| mouse c-abl IV | SALIKKKKKMAP | 45 |
| influenza virus NS1 | DRLRR | 46 |
| influenza virus NS1 | PKQKKRK | 47 |
| Hepatitis virus delta | RKLKKKIKKL | 48 |
| antigen | | |
| mouse Mx1 protein | REKKKELKRR | 49 |
| human poly (ADP- | KRKGDEVDGVDEVAKKKSKK | 50 |
| ribose) polymerase | | |
| steroid hormone | RKCLQAGMNLEARKTKK | 51 |
| receptors (human) | | |
| glucocorticoid | | |

In some cases, the engineered nuclease system further comprises a single- or double stranded DNA repair template. In some cases, the engineered nuclease system further comprises a single-stranded DNA repair template. In some cases, the engineered nuclease system further comprises a double-stranded DNA repair template. In some cases, the single- or double-stranded DNA repair template may comprise from 5′ to 3′: a first homology arm comprising a sequence of at least 20 nucleotides 5′ to said target deoxyribonucleic acid sequence, a synthetic DNA sequence of at least 10 nucleotides, and a second homology arm comprising a sequence of at least 20 nucleotides 3′ to said target sequence.

In some cases, the first homology arm comprises a sequence of at least 40, at least 50, at least 60, at least 70, at least 80, at least 90, at least 100, at least 110, at least 120, at least 130, at least 140, at least 150, at least 175, at least 200, at least 250, at least 300, at least 400, at least 500, at least 750, or at least 1000 nucleotides. In some cases, the second homology arm comprises a sequence of at least 40, at least 50, at least 60, at least 70, at least 80, at least 90, at least 100, at least 110, at least 120, at least 130, at least 140, at least 150, at least 175, at least 200, at least 250, at least 300, at least 400, at least 500, at least 750, or at least 1000 nucleotides.

In some cases, the first and second homology arms are homologous to a genomic sequence of a prokaryote. In some cases, the first and second homology arms are homologous to a genomic sequence of a bacteria. In some cases, the first and second homology arms are homologous to a genomic sequence of a fungus. In some cases, the first and second homology arms are homologous to a genomic sequence of a eukaryote.

In some cases, the engineered nuclease system further comprises a DNA repair template. The DNA repair template may comprise a double-stranded DNA segment. The double-stranded DNA segment may be flanked by one single-stranded DNA segment. The double-stranded DNA segment may be flanked by two single-stranded DNA segments. In some cases, the single-stranded DNA segments are conjugated to the 5′ ends of the double-stranded DNA segment. In some cases, the single stranded DNA segments are conjugated to the 3′ ends of the double-stranded DNA segment.

In some cases, the single-stranded DNA segments have a length from 1 to 15 nucleotide bases. In some cases, the single-stranded DNA segments have a length from 4 to 10 nucleotide bases. In some cases, the single-stranded DNA segments have a length of 4 nucleotide bases. In some cases, the single-stranded DNA segments have a length of 5 nucleotide bases. In some cases, the single-stranded DNA segments have a length of 6 nucleotide bases. In some cases, the single-stranded DNA segments have a length of 7 nucleotide bases. In some cases, the single-stranded DNA segments have a length of 8 nucleotide bases. In some cases, the single-stranded DNA segments have a length of 9 nucleotide bases. In some cases, the single-stranded DNA segments have a length of 10 nucleotide bases.

In some cases, the single-stranded DNA segments have a nucleotide sequence complementary to a sequence within the spacer sequence. In some cases, the double-stranded DNA sequence comprises a barcode, an open reading frame, an enhancer, a promoter, a protein-coding sequence, a miRNA coding sequence, an RNA coding sequence, or a transgene.

In some cases, the engineered nuclease system further comprises a source of Mg²⁺.

In some cases, the guide RNA comprises a hairpin comprising at least 8 base-paired ribonucleotides. In some cases, the guide RNA comprises a hairpin comprising at least 9 base-paired ribonucleotides. In some cases, the guide RNA comprises a hairpin comprising at least 10 base-paired ribonucleotides. In some cases, the guide RNA comprises a hairpin comprising at least 11 base-paired ribonucleotides. In some cases, the guide RNA comprises a hairpin comprising at least 12 base-paired ribonucleotides.

In some cases, the endonuclease comprises a sequence at least 70% identical to a variant of any one of SEQ ID NOs: 1-3, 11, 13, or 15, or a variant thereof. In some cases, the endonuclease comprises a sequence at least 75% identical to a variant of any one of SEQ ID NOs: 1-3, 11, 13, or 15, or a variant thereof. In some cases, the endonuclease comprises a sequence at least 80% identical to a variant of any one of SEQ ID NOs: 1-3, 11, 13, or 15, or a variant thereof. In some cases, the endonuclease comprises a sequence at least 85% identical to a variant of any one of SEQ ID NOs: 1-3, 11, 13, or 15, or a variant thereof. In some cases, the endonuclease comprises a sequence at least 90% identical to a variant of any one of SEQ ID NOs: 1-3, 11, 13, or 15, or a variant thereof. In some cases, the endonuclease comprises a sequence at least 95% identical to a variant of any one of SEQ ID NOs: 1-3, 11, 13, or 15, or a variant thereof.

In some cases, sequence may be determined by a BLASTP. CLUSTALW. MUSCLE, or MAFFT algorithm, or a CLUSTALW algorithm with the Smith-Waterman homology search algorithm parameters. The sequence identity may be determined by said BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

In one aspect, the present disclosure provides an engineered guide RNA comprising a DNA-targeting segment. In some cases, the DNA-targeting segment comprises a nucleotide sequence that is complementary to a target sequence. In some cases, the target sequence is in a target DNA molecule. In some cases, the engineered guide RNA comprises a protein-binding segment. In some cases, the protein-binding segment comprises two complementary stretches of nucleotides. In some cases, the two complementary stretches of nucleotides hybridize to form a double-stranded RNA (dsRNA) duplex. In some cases, the two complementary stretches of nucleotides are covalently linked to one another with intervening nucleotides. In some cases, the engineered guide ribonucleic acid polynucleotide is capable of forming a complex with an endonuclease. In some cases, the endonuclease has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-15. In some cases, the complex targets the target sequence of the target DNA molecule. In some cases, the DNA-targeting segment is positioned 3′ of both of the two complementary stretches of nucleotides.

In some cases, the double-stranded RNA (dsRNA) duplex comprises at least 8 ribonucleotides. In some cases, the double-stranded RNA (dsRNA) duplex comprises at least 9 ribonucleotides. In some cases, the double-stranded RNA (dsRNA) duplex comprises at least 10 ribonucleotides. In some cases, the double-stranded RNA (dsRNA) duplex comprises at least 11 ribonucleotides. In some cases, the double-stranded RNA (dsRNA) duplex comprises at least 12 ribonucleotides.

In some cases, the deoxyribonucleic acid polynucleotide encodes the engineered guide ribonucleic acid polynucleotide.

In one aspect, the present disclosure provides a nucleic acid comprising an engineered nucleic acid sequence. In some cases, the engineered nucleic acid sequence is optimized for expression in an organism. In some cases, the nucleic acid encodes an endonuclease. In some cases, the endonuclease is a Cas endonuclease. In some cases, the endonuclease is a class 2 endonuclease. In some cases, the endonuclease is a class2, type V Cas endonuclease. In some cases, the endonuclease is a class2, type V, novel subtype Cas endonuclease. In some cases, the endonuclease is derived from an uncultivated microorganism. In some cases, the organism is not the uncultivated organism.

In some cases, the endonuclease comprises a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1-15.

In some cases, the endonuclease may comprise a variant having one or more nuclear localization sequences (NLSs). The NLS may be proximal to the N- or C-terminus of the endonuclease. The NLS may be appended N-terminal or C-terminal to any one of SEQ ID NOS: 36-51, or to a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 36-51.

In some cases, the organism is prokaryotic. In some cases, the organism is bacterial. In some cases, the organism is eukaryotic. In some cases, the organism is fungal. In some cases, the organism is a plant. In some cases, the organism is mammalian. In some cases, the organism is a rodent. In some cases, the organism is human.

In one aspect, the present disclosure provides an engineered vector. In some cases, the engineered vector comprises a nucleic acid sequence encoding an endonuclease. In some cases, the endonuclease is a Cas endonuclease. In some cases, the endonuclease is a class 2 Cas endonuclease. In some cases, the endonuclease is a class 2, type V Cas endonuclease. In some cases, the endonuclease is a class2, type V, novel subtype Cas endonuclease. In some cases, the endonuclease is derived from an uncultivated microorganism.

In some cases, the engineered vector comprises a nucleic acid described herein. In some cases, the nucleic acid described herein is a deoxyribonucleic acid polynucleotide described herein. In some cases, the vector is a plasmid, a minicircle, a CELiD, an adeno-associated virus (AAV) derived virion, or a lentivirus.

In one aspect, the present disclosure provides a cell comprising a vector described herein.

In one aspect, the present disclosure provides a method of manufacturing an endonuclease. In some cases, the method comprises cultivating the cell.

In one aspect, the present disclosure provides a method for binding, cleaving, marking, or modifying a double-stranded deoxyribonucleic acid polynucleotide. The method may comprise contacting the double-stranded deoxyribonucleic acid polynucleotide with an endonuclease. In some cases, the endonuclease is a Cas endonuclease. In some cases, the endonuclease is a class 2 Cas endonuclease. In some cases, the endonuclease is a class 2, type V Cas endonuclease. In some cases, the endonuclease is a class2, type V, novel subtype Cas endonuclease. In some cases, the endonuclease is in complex with an engineered guide RNA. In some cases, the engineered guide RNA is configured to bind to the endonuclease. In some cases, the engineered guide RNA is configured to bind to the double-stranded deoxyribonucleic acid polynucleotide. In some cases, the engineered guide RNA is configured to bind to the endonuclease and to the double-stranded deoxyribonucleic acid polynucleotide. In some cases, the double-stranded deoxyribonucleic acid polynucleotide comprises a protospacer adjacent motif (PAM).

In some cases, the double-stranded deoxyribonucleic acid polynucleotide comprises a first strand comprising a sequence complementary to a sequence of the engineered guide RNA and a second strand comprising the PAM. In some cases, the PAM is directly adjacent to the 5′ end of the sequence complementary to the sequence of the engineered guide RNA. In some cases, the endonuclease is not a Cpf1 endonuclease or a Cms1 endonuclease. In some cases, the endonuclease is derived from an uncultivated microorganism. In some cases, the double-stranded deoxyribonucleic acid polynucleotide is a eukaryotic, plant, fungal, mammalian, rodent, or human double-stranded deoxyribonucleic acid polynucleotide.

In one aspect, the present disclosure provides a method of modifying a target nucleic acid locus. The method may comprise delivering to the target nucleic acid locus the engineered nuclease system described herein. In some cases, the endonuclease is configured to form a complex with the engineered guide ribonucleic acid structure. In some cases, the complex is configured such that upon binding of the complex to the target nucleic acid locus, the complex modifies the target nucleic acid locus.

In some cases, modifying the target nucleic acid locus comprises binding, nicking, cleaving, or marking said target nucleic acid locus. In some cases, the target nucleic acid locus comprises deoxyribonucleic acid (DNA) or ribonucleic acid (RNA). In some cases, the target nucleic acid comprises genomic DNA, viral DNA, viral RNA, or bacterial DNA. In some cases, the target nucleic acid locus is in vitro. In some cases, the target nucleic acid locus is within a cell. In some cases, the cell is a prokaryotic cell, a bacterial cell, a eukaryotic cell, a fungal cell, a plant cell, an animal cell, a mammalian cell, a rodent cell, a primate cell, or a human cell.

In some cases, delivery of the engineered nuclease system to the target nucleic acid locus comprises delivering the nucleic acid described herein or the vector described herein. In some cases, delivery of engineered nuclease system to the target nucleic acid locus comprises delivering a nucleic acid comprising an open reading frame encoding the endonuclease. In some cases, the nucleic acid comprises a promoter. In some cases, the open reading frame encoding the endonuclease is operably linked to the promoter.

In some cases, delivery of the engineered nuclease system to the target nucleic acid locus comprises delivering a capped mRNA containing the open reading frame encoding the endonuclease. In some cases, delivery of the engineered nuclease system to the target nucleic acid locus comprises delivering a translated polypeptide. In some cases, delivery of the engineered nuclease system to the target nucleic acid locus comprises delivering a deoxyribonucleic acid (DNA) encoding the engineered guide RNA operably linked to a ribonucleic acid (RNA) pol III promoter.

In some cases, the endonuclease induces a single-stranded break or a double-stranded break at or proximal to the target locus. In some cases, the endonuclease induces a staggered single stranded break within or 3′ to said target locus.

In some cases, effector repeat motifs are used to inform guide design of MG nucleases. For example, the processed gRNA in Type V systems consists of the last 20-22 nucleotides of a CRISPR repeat. This sequence may be synthesized into a crRNA (along with a spacer) and tested in vitro, along with the synthesized nucleases, for cleavage on a library of possible targets. Using this method, the PAM may be determined. In some cases, Type V enzymes may use a “universal” gRNA. In some cases, Type V enzymes may need a unique gRNA.

Systems of the present disclosure may be used for various applications, such as, for example, nucleic acid editing (e.g., gene editing), binding to a nucleic acid molecule (e.g., sequence-specific binding). Such systems may be used, for example, for addressing (e.g., removing or replacing) a genetically inherited mutation that may cause a disease in a subject, inactivating a gene in order to ascertain its function in a cell, as a diagnostic tool to detect disease-causing genetic elements (e.g. via cleavage of reverse-transcribed viral RNA or an amplified DNA sequence encoding a disease-causing mutation), as deactivated enzymes in combination with a probe to target and detect a specific nucleotide sequence (e.g. sequence encoding antibiotic resistance int bacteria), to render viruses inactive or incapable of infecting host cells by targeting viral genomes, to add genes or amend metabolic pathways to engineer organisms to produce valuable small molecules, macromolecules, or secondary metabolites, to establish a gene drive element for evolutionary selection, to detect cell perturbations by foreign small molecules and nucleotides as a biosensor.

### EXAMPLES

In accordance with IUPAC conventions, the following abbreviations are used throughout the examples:
A=adenine
C=cytosine
G=guanine
T=thymine
R=adenine or guanine
Y=cytosine or thymine
S=guanine or cytosine
W=adenine or thymine
K=guanine or thymine
M=adenine or cytosine
B=C, G, or T
D=A, G, or T
H=A. C, or T
V=A, C, or G

### Example 1—a Method of Metagenomic Analysis for New Proteins

Metagenomic samples were collected from sediment, soil, and animals. Deoxyribonucleic acid (DNA) was extracted with a Zymobiomics DNA mini-prep kit and sequenced on an Illumina HiSeq® 2500. Samples were collected with consent of property owners. Additional raw sequence data from public sources included animal microbiomes, sediment, soil, hot springs, hydrothermal vents, marine, peat bogs, permafrost, and sewage sequences. Metagenomic sequence data was searched using Hidden Markov Models generated based on known Cas protein sequences including class II type V Cas effector proteins to identify new Cas effectors. Novel effector proteins identified by the search were aligned to known proteins to identify potential active sites. This metagenomic workflow resulted in the delineation of the MG90, MG118, MG119, MG120, MG126, and MG127 families described herein.

### Example 2—Discovery of MG90, MG118, MG119, MG120, MG126, and MG127 Families of CRISPR Systems

Analysis of the data from the metagenomic analysis of Example 1 revealed new clusters of previously undescribed putative CRISPR systems comprising 9 families (MG90, MG118, MG119, MG120, MG126, and MG127). The corresponding protein and nucleic acid sequences for these new enzymes and their exemplary subdomains are presented as SEQ ID NOs: 1-15.

### Example 3—Template DNA for Transcription and Translation

E. coli codon optimized sequences of all MG VU and CasPhi nucleases were ordered (Twist Biosciences) in a plasmid with a T7 promoter. Linear templates were amplified from the plasmids by PCR to include the T7 and nuclease sequence. Minimal array linear templates were amplified from sequences composed of a T7 promoter, native repeat, universal spacer, and native repeat, flanked by adapter sequences for amplification. The universal spacer matches the spacer in an 8N target library, where there are &N mixed bases adjacent to the spacer for PAM determination. Three intergenic sequences near the ORF or CRISPR array were identified from the metagenomic contigs and ordered as gBlocks with flanking adapter sequences for amplification (Integrated DNA Technologies).

### Example 4—In Vitro Transcription of crRNA, Minimal Arrays, and sgRNA

RNA was produced by in vitro transcription using HiScribe™ T7 High Yield RNA Synthesis Kit and purified using the Monarch R RNA Cleanup Kit (New England Biolabs Inc.). Templates for T7 transcription varied. For crRNA, DNA oligos were designed with a T7 promoter, trimmed native repeat, and universal spacer. For minimal arrays the same templates as described above were used. For sgRNA. DNA ultramers were designed with a T7 promoter, trimmed tracrRNA, GAAA tetraloop, trimmed native repeat, and universal spacer. Minimal array templates were amplified with adapter primers. The crRNA and sgRNA templates were ordered as reverse complements and annealed with a primer with the T7 promoter sequence in 1×IDT duplex buffer at 95° C., for two minutes followed by cooling to 22° C., at 0.1° C./second to produce a hybrid ds/ssDNA substrate suitable for transcription. After transcription, but prior to cleaning, each reaction was treated with DNAse I and incubated at 37° C., for 15 minutes. All transcription products were verified for yield and purity via RNA Tapestation or via a denaturing urea PAGE gel.

### Example 5—TXTL Expression

Nucleases, intergenic sequences, and minimal arrays were expressed in transcription-translation reaction mixtures using myTXTL® Sigma 70 Master Mix Kit (Arbor Biosciences) The final reaction mixtures contained 5 nM nuclease DNA template, 12 nM intergenic DNA template, 15 nM minimal array DNA template, 0.1 nM pTXTL-P70a-T7map, and 1× of my TXTL® Sigma 70 Master Mix. The reactions were incubated at 29° C., for 16 hours then stored at 4° C.

### Example 6—PURExpress Expressions

10 nM of nuclease PCR templates were expressed at 37° C., for 3 hours with PURExpress® In Vitro Protein Synthesis Kit (New England Biolabs Inc.) for cleavage with in vitro transcribed RNA. These reactions were used to test in vitro cleavage with 50 nM sgRNA or minimal array RNA following the same procedure as described in the cleavage reactions section.

### Example 7—E. coli Expressions

Plasmids encoding the effector, intergenic sequence from the genomic contig, native repeat, and universal spacer sequences with a T7 promoter were transformed into BL21 DE3 or T7 Express lysY/Iq and cultured at 37° C., in 60 mL terrific broth media supplemented with 100 μg/mL of ampicillin. Expression was induced with 0.4 mM IPTG after cultures reached OD₆₀₀ₙₘ of 0.5 and incubated at 16° C., overnight. 25 mL of cells were pelleted by centrifugation and resuspended in 1.5 mL, of lysis buffer (20 mM Tris-HCl, 500 mM NaCl, 1 mM TCEP, 5% glycerol, 10 mM MgCl2 pH 7.5 with Pierce Protease Inhibitor, (Thermo Scientific|M)). Cells were then lysed by sonication. Supernatant and cell debris were separated by centrifugation.

### Example 8—Cleavage Reactions

Plasmid library DNA cleavage reactions were carried out by mixing 5 nM of the target library, a 5-fold dilution of the TXTL or PURExpress expressions. 10 nM Tris-HCl, 10 nM MgCl₂, and 100 mM NaCl at 37° C., for 2 hours. For reactions with E, coli expressions, 10 μL of the clarified lysate was added. Reactions were stopped and cleaned with HighPrep™ PCR clean up beads (MAGBIO Genomics, Inc.) and eluted in Tris EDTA pH 8.0 buffer 3 nM of the cleavage product ends were blunted with 3.33 μM dNTPs, IX T4 DNA ligase buffer, and 0.167 U/μL of Klenow Fragment (New England Biolabs Inc.) at 25° C., for 15 minutes. 1.5 nM of the cleavage products were ligated with 150 nM adapters, 1×T4 DNA ligase buffer (New England Biolabs Inc.), 20 U/μL T4 DNA ligase (New England Biolabs Inc.) at room temperature for 20 minutes. The ligated products were amplified by PCR with NGS primers and sequenced by NGS to obtain the PAM.

### Example 9—RNAseq Library Prep of Intergenic Enrichment from TXTL and E, Coli Lysates

RNA is extracted from TXTL and cell lysate expressions following the Quick-RNA™ Miniprep Kit (Zymo Research) and eluted in 30-50 μL of water. The total concentration of the transcripts were measured on a Nanodrop. Tapestation, and Qubit.

100 ng-1 ug of total RNA from each sample were prepped for RNA sequencing using the NEBNext Small RNA Library Prep Set for Illumina (New England Biolabs Inc.). Amplicons between 150-300 bp were quantified by Tapestation and Qubit and pooled to a final concentration of 4 nM A final concentration of 12.5 pM was loaded into a MiSeq V3 kit and sequenced in a Miseq system (Illumina) for 176 total cycles. The RNAseq reads were used to identify the tracr sequence of the genes.

### Example 10—Predicted RNA Folding

Predicted RNA folding of the active single RNA sequence was computed at 37 using the method of Andronescu 2007. The shading of the bases corresponds to the probability of base pairing of that base.

### Example 11—In Vitro Cleavage Efficiency (Prophetic)

The protein is expressed in E, coli protease deficient B strain under T7 inducible promoter, the cells are lysed using sonication, and the His-tagged protein of interest is purified using HisTrap FF (GE Lifescience) Ni-NTA affinity chromatography on the AKTA Avant FPLC (GE Lifescience). Purity is determined using densitometry in ImageLab software (Bio-Rad) of the protein bands resolved on SDS-PAGE and InstantBlue Ultrafast (Sigma-Aldrich) coomassie stained acrylamide gels (Bio-Rad). The protein is desalted in a storage buffer composed of 50 mM Tris-HCl, 300 mM NaCl. 1 mM TCEP. 5% glycerol; pH 7.5 and stored at −80° C.

A target DNA is constructed that contains a spacer sequence and a PAM determined via NGS. In the case of degenerate bases in the PAM a single representative PAM is chosen for testing. The target DNA is 2200 bp of linear DNA derived from a plasmid via PCR amplification. The PAM and spacer are located 700 bp from one end. Successful cleavage results in fragments of 700 and 1500 bp.

The target DNA, in vitro transcribed single RNA, and purified recombinant protein are combined in cleavage buffer (10 mM Tris. 100 mM NaCl. 10 mM MgCl2) with an excess of protein and RNA and incubated for 5 min to 3 hours, for example 1 h. The reaction is stopped via addition of RNAse A and incubation at 60° C. The reaction is resolved on a 1.2% TAE agarose gel and the fraction of cleaved target DNA is quantified in ImageLab software.

### Example 12—Activity in E. coli (Prophetic)

For testing of nuclease activity in bacterial cells, strains are constructed with genome sequences containing the target spacer and corresponding PAM sequence specific to the enzyme of interest. Engineered strains are then transformed with the nuclease of interest and transformants are then subsequently made chemocompetent and transformed with 50 ng of single guides either specific to the target sequence (on target) or non-specific to the target (off target). After heat shock, transformations are recovered in SOC for 2 h at 37° C., and nuclease efficiency is determined by a 5-fold dilution series grown on induction media. Colonies are quantified from the dilution series in triplicate.

### Example 13—Activity in Mammalian Cells (Prophetic)

To show targeting and cleavage activity in mammalian cells, the protein sequences are cloned into 2 mammalian expression vectors, one with a C-terminal SV40 NLS and a 2A-GFP tag and one with no GFP tag and 2 NLS sequences, one on the N-terminus and one on the C-terminus. Alternative NLS sequences that can also be used. The DNA sequence for the protein can be the native sequence, the E, coli codon optimized sequence, or the mammalian codon optimized sequence. The single guide RNA sequence with a gene target of interest is also cloned into a mammalian expression vector. The two plasmids are cotransfected into HEK293T cells. 72 h after co-transfection of the expression plasmid and sgRNA targeting plasmid, the DNA is extracted and used for the preparation of an NGS-library. Percent NHEJ is measured via indels in the sequencing of the target site to demonstrate the targeting efficiency of the enzyme in mammalian cells. At least 10 different target sites are chosen for testing each protein's activity.

TABLE 2
Protein and nucleic acid sequences referred to herein

| | SEQ | | | | | |
|---|---|---|---|---|---|---|
| | ID | | | | Other | |
| Cat. | NO: | Description | Type | Organism | Information | Sequence |
| MG120 | 1 | MG120-10 | protein | unknown | uncultivated | MTESAEQDSGRFVLELPLRVHS |
| Effectors | | Effector | | | organism | GQDKTLRTRFEAGRQIYNACLG |
| | | | | | | ECLRRLDLMRESKDYQHARAMP |
| | | | | | | RGKDRTKAFRDLDKRFEFRKYD |
| | | | | | | LFHWTTQFTQSWLNQHVDSQTT |
| | | | | | | KRLMERAFNATREYMEGKRGRP |
| | | | | | | RFRGRRGLNSLESTSNTNGIKW |
| | | | | | | RDGTIKWIGLEIPAIVDPEDPV |
| | | | | | | QMHGLSSEIKYCRIVKRDLNGK |
| | | | | | | RRWFIQLVLVGEPLQRFELGKG |
| | | | | | | DVGLDIGPSTIAAVGTDDALLV |
| | | | | | | QFCAELSEDQRETRRLQRKIDR |
| | | | | | | QRRANNPDCYDGRGRAIKGKHP |
| | | | | | | HKRSLRMRETEAQLAEVKRKLA |
| | | | | | | AYRKSLHGELAHRILEMGDSIK |
| | | | | | | TEKLSYKALQRIWGKSVGTRAP |
| | | | | | | GMFVDLLRRKAESAGGEFVEFS |
| | | | | | | TYHTRLSQTCHQCGAIVKKSLS |
| | | | | | | VRWHNCDCGITAQRDLYSAFLA |
| | | | | | | SCVIEGSLDADLARERWSSAGP |
| | | | | | | LLQTAFKETQLASSGASVPASF |
| | | | | | | GLGQSQSESSVKRGQHATKVAD |
| | | | | | | VVTAQGIESARAAERLQNARES |
| | | | | | | LLSDL |
| MG118 | 2 | MG118-2 | protein | unknown | uncultivated | MTMTTVKDIKQEHFQSKSFLDK |
| Effectors | | Effector | | | organism | DIKMAGRALAKYGEAACIEYLQ |
| | | | | | | NAPAAQGNLKRPTKCNIIAVSR |
| | | | | | | PLAEWPALLASANIQKHVYSLT |
| | | | | | | FSELEEFAEAFSKPSKGSRESI |
| | | | | | | LKVIETHGYSSAQGLNLMIGNA |
| | | | | | | ISTYKGVLKKVDNRNKKKAERI |
| | | | | | | ARINDSRKSNVNYVPLEFVPES |
| | | | | | | ALDDEGHLINPPGINRNLFCYQ |
| | | | | | | GLKLRPYAPTILPKEGEKTRHI |
| | | | | | | HQKLNLPLAYDAYDRDPDAPIV |
| | | | | | | CGALDRLSVPKGELGYVPEHPI |
| | | | | | | GWKEPLLQRHNLKKKGRVRLGG |
| | | | | | | NKSFKASQAIIVIISVGDDWVA |
| | | | | | | FDARGLLRQIRRLNLCPKEEEL |
| | | | | | | TVETILSKFTDSPVINPNEGVV |
| | | | | | | TFMVDPALVVGFGKKPVHRNST |
| | | | | | | KKRLLDLCSTSDVALVSVDLNK |
| | | | | | | NNTFAYRISRVRKNGEELEPEI |
| | | | | | | ITFGCYDCSTKEFVSLRSAHDM |
| | | | | | | AEEDYYNQALSSLSLEDQALIK |
| | | | | | | ASKNSDLVKAKVCERLSISKND |
| | | | | | | IDWDSMKYETSFISKFCSEHGI |
| | | | | | | EDDDLLKIVERYKRGSRKGQIK |
| | | | | | | SKKNAYDGKVIRKIVALPSDVN |
| | | | | | | ERLQKARWKFQRESSSFKRLAE |
| | | | | | | RKRQVANAYVNKVIVQAKKLSQ |
| | | | | | | CDVVAFVVEDGVNKFHGGGVRE |
| | | | | | | IGLDSLLRPKKEGRWLIQAFYR |
| | | | | | | SITDLAPNKGIDVLMIDPRWTS |
| | | | | | | TTCIVCGNVDKANRSSDNKEEF |
| | | | | | | ICTNTKCGDKRNADIEVATENI |
| | | | | | | QKCALTGRAMPGPEKRERSSDG |
| | | | | | | KKSRTSRRTKAHQIIDEIHNAA |
| | | | | | | E |
| MG90 | 3 | MG90-4 | protein | unknown | uncultivated | MPTKKKTYSSARKSSKVPTRNF |
| Effectors | | Effector | | | organism | VYGVGCKGEQPTPQDVIRDQLW |
| | | | | | | EAHQYKVALIRNGLARIDAENA |
| | | | | | | ILCEVSPRLGELQRQSDDLTEK |
| | | | | | | IKTMRAELKSPREDRADSPESA |
| | | | | | | AIQAVVDERKAVYEEMKKVRKD |
| | | | | | | LFALTSTYNADLNRKLEQGLLE |
| | | | | | | LAVEKDPAFAPLLDGCPEGENP |
| | | | | | | KEFKKRVKRATSIIGPRNPKRV |
| | | | | | | ALNARLTEQMLSDDSVPDTWKR |
| | | | | | | IKEVQQECHKKNLQARKDSKCF |
| | | | | | | SGTYQAVDAAVRDAFQKSYHPR |
| | | | | | | VPKFNRTGKVGIQLTGERTVNG |
| | | | | | | KTVKGLPVSEVESPDNQFSLEL |
| | | | | | | LPDHRVYGARKKHNRKEYPVMA |
| | | | | | | RARICVARGVRVETLVVMHRPL |
| | | | | | | PEDGVIKWAYLEVKRVGLKTRY |
| | | | | | | ELRLVIEYFPKPRPKVTKKPLL |
| | | | | | | AVNEGYRAMADGTIRLATTWDG |
| | | | | | | EEVGGITLPSHIKRDEEFSRRL |
| | | | | | | DGHADKRENTAQKELGSWKKTV |
| | | | | | | KDMPDELKEKIRNSGQWRAHGK |
| | | | | | | LAVATHLLVRTYVPESETTVRR |
| | | | | | | LWHTWRVERLEQGLDLEDERPF |
| | | | | | | LENWFREQGVRSRNARFALYME |
| | | | | | | WWRRKDVHLVDLAKGFKARYKR |
| | | | | | | RIKEIVRVQVARWRDSYEVIAS |
| | | | | | | NEWDMRKTARKPKPENDTRTKR |
| | | | | | | EINANGVRQIGGPGIVNLYLKE |
| | | | | | | VFGDRRMTTDSKGASKIHFGCG |
| | | | | | | GEGTGIETDLRCNKCGRGEDQD |
| | | | | | | INAARHTWKEANGTLNGTPPKK |
| | | | | | | APKRKRSSRLAETTSSV |
| MG90 | 4 | MG90-9 | protein | unknown | uncultivated | MTARRQRSTEVRIYKYGVLAPS |
| Effectors | | Effector | | | organism | SDARSHLDEELFRANQYHNELI |
| | | | | | | AIERTRRERFAAVRAEAVPELQ |
| | | | | | | GVEGRLMAIESELDVTFEAHRG |
| | | | | | | GDDAASGRLAALKEERATLTAR |
| | | | | | | AKQLRDEFKALLAPGQQAFDER |
| | | | | | | TSGAPAALLRELDRAGRALKEA |
| | | | | | | KKIRRNPEVPILRRRLDELKRQ |
| | | | | | | RRSYLPSPHRKAEANETGLNQM |
| | | | | | | MDEDWPDAWKRVARADAEAVVA |
| | | | | | | IKKARAQKSVGAPTSRLIERAV |
| | | | | | | EAVRRDSKMEPRERRERGEGQI |
| | | | | | | GGQVPDADGTTKIFSAKNGLIR |
| | | | | | | IGEKVSGSGRFAKRAVELRVGE |
| | | | | | | WWIALSARWHRELPADARITWA |
| | | | | | | WLKVRRDGWTKRYELQLTVESE |
| | | | | | | RHSTSRREVAQDGKVRVRLGWA |
| | | | | | | SIDDAPQVLRPGESPPDARYRP |
| | | | | | | LIVATAEDERGTVTECVLPRSE |
| | | | | | | IERLNHREANLRRISDLHYNET |
| | | | | | | HKIFQSVVRRWLERHPESVPTW |
| | | | | | | ITERPVVRGEARRSMVDTMHLW |
| | | | | | | RSHEALARASFRWCREVLGEER |
| | | | | | | VNDLWLRWRDERKARGLDLFGR |
| | | | | | | IGEFTRWARATGVVDSRELLVL |
| | | | | | | YFEWWRRKELHLADWGTRQTRG |
| | | | | | | AERARAQRYRLWGIDLSKLYAT |
| | | | | | | LTLEDIDLSRTAQDHGARDRAP |
| | | | | | | NPVDSRVRSQQRDAAPSVMRAA |
| | | | | | | LLEVEGKDRTTILKRERSGGED |
| | | | | | | RPGTARRAEKKPGHERRESIDD |
| | | | | | | RAAE |
| MG90 | 5 | MG90-10 | protein | unknown | uncultivated | MATKVYKYGARPVRCPLVDEQM |
| Effectors | | Effector | | | organism | RLGHRYYNALVEAENDRRKEGR |
| | | | | | | GGDTPPAPPHDDCECKECREHW |
| | | | | | | KILRKRMRDLPFIDVKPLRAEY |
| | | | | | | AKKGLYWGTYLHVEKDEDAAKM |
| | | | | | | KTNIFRTVKERSWHRGGLIAVQ |
| | | | | | | IQRATHDRNGGKTMFQISDAPD |
| | | | | | | SRSGRRNRGGRGRALVKMRVGS |
| | | | | | | DGRVPVFCTPIKIERHRLMTGS |
| | | | | | | PTWVKLTKRTDAERETWEVHVT |
| | | | | | | CAGVEETPGAERGVVAVDCGWR |
| | | | | | | KLPDGSWRLGYARGDQGAVSEF |
| | | | | | | VLPATWSELAHRADRIRSHRDE |
| | | | | | | RLNALKAEDKRFSRCQSCLGVV |
| | | | | | | RAVRKMREEGAPVDKDDEIKEW |
| | | | | | | LRRDRHLWQYEVGCRRRSVARR |
| | | | | | | RDEMRKWVRRLRDQYREVIVKD |
| | | | | | | SNHKRMKESSGLHQAARRQGHH |
| | | | | | | GSPGELVEALRQVFGADAMHVV |
| | | | | | | SAVHTTNTCVECGHKNNFGPER |
| | | | | | | EVICEGCGVVDDRDSVSTQNMI |
| | | | | | | RLFADGQYSGPTARKTEAKFSK |
| | | | | | | RHRTSEEGVSASPVA |
| MG90 | 6 | MG90-11 | protein | unknown | uncultivated | MKVYKYGITSDLDDQITAQMKA |
| Effectors | | Effector | | | organism | GHDYYNQLVEAENRRRYDLWFG |
| | | | | | | GATPAPPHTGCSCKDCRDHYRN |
| | | | | | | LTPPKPPHTPCTQKPCTACNAF |
| | | | | | | WQPENDASWLDLKPLRKQRPDG |
| | | | | | | LYWGTYLLIERAFMQAVKTTKI |
| | | | | | | LDRVRFRPYRRSSTVCGVQIQK |
| | | | | | | NKRLDAFFQIEARHDSRKGKRK |
| | | | | | | GRRHRFRLRVGSQGRTPIWSDW |
| | | | | | | INIELHRPWSGDIKWIAVVRAA |
| | | | | | | NGKCSLQITCNNENARTDNAAH |
| | | | | | | GAVAVDISWRKLQNGSWRLAMA |
| | | | | | | RDLNGNLDELTLPPEWAELSAR |
| | | | | | | ADRIRGHRDKRLNELAAVDSRE |
| | | | | | | AVCKHPSGVERRARKLGIDQGD |
| | | | | | | AIWDWVARDRHLDQYEAGCRRR |
| | | | | | | SIARRRNDMRVWVRQLRRNYAT |
| | | | | | | VIIKDTSHKKLKEKKDLHQGAR |
| | | | | | | RQGQRAAPGEIVEQLRRVFGKG |
| | | | | | | MQLVPAPMTNHVLCGPGHRAVE |
| | | | | | | VGADLMLVCERCGARHDRDHVS |
| | | | | | | TQNMLALWSAGEGVDPPARRTG |
| | | | | | | GRFANQHGSSASP |
| MG90 | 7 | MG90-12 | protein | unknown | uncultivated | MGQLLNNTDRLIVVQRRSKPKS |
| Effectors | | Effector | | | organism | SWKIVARFATVAEAEVTKERYA |
| | | | | | | ADERREVEQIKNGEKKPDKKKK |
| | | | | | | PQPSETAEYRVYYDTITRVYRY |
| | | | | | | GLRSPDVERDVDQQMFQGHVYR |
| | | | | | | NSLVAIYKASREEYRALLNTVG |
| | | | | | | DVDAAGQHKREKAAIVEQLKQQ |
| | | | | | | IKTVRKRSRARAEPPELKARLK |
| | | | | | | AAKAELTLATNSYKVLVAKADE |
| | | | | | | AGLYTKGKKAIDATKGRAIKEA |
| | | | | | | RRVARTEQDLFWGTYGRREEFA |
| | | | | | | ARANKGHYDPKFHRWDYGDGST |
| | | | | | | WLAVQLQKGLSLSDLFSAKNRY |
| | | | | | | LQIAPIDLLPDENDPRVWNKSV |
| | | | | | | RKGERRRRSRTLVRVCIGSTSG |
| | | | | | | KPVYATFPLELHRPLPANSKVK |
| | | | | | | WAVVIRERVGRGQDCDKWSLHL |
| | | | | | | SMEIPKKPVVRRPGVVGVNLGW |
| | | | | | | RLKQSRKAGGVSILRVAYWADD |
| | | | | | | QGNHGEVLLDDAIWHRDNKAKE |
| | | | | | | GGYLGHARHLRSIRDKAFNAAR |
| | | | | | | DDLGSWLRGRRKRRQYIPKWLR |
| | | | | | | TGAKNLGNWRSARRLAKLVWKW |
| | | | | | | KDNRFADDVEIYRRLEGWRSIG |
| | | | | | | ADGKPFYEGWRKQDKHLHNWEA |
| | | | | | | HSARRGRNKRKNTCQHFAKRLA |
| | | | | | | TLYGTVVVTQMDYAKAARRAGP |
| | | | | | | GEEKESHDAARRQRQVAAPSRL |
| | | | | | | RDAIQNACSTHGSDENALPVRT |
| | | | | | | DWCHVCGTPNLWASEKSKIEDI |
| | | | | | | KHTCKGCGNVIDQDHNAAEIVW |
| | | | | | | RIWTDNENKKAA |
| MG90 | 8 | MG90-13 | protein | unknown | uncultivated | MAKKHKNQSKATRVYAFGAYPS |
| Effectors | | Effector | | | organism | VENSELVDDIFYRAWRERNDLI |
| | | | | | | RVELDYRKRRDEIVREYSLKVR |
| | | | | | | NLEAEHQCLTQEIADREGEVNT |
| | | | | | | QSIQDRKRCLAPELKGLRKQLK |
| | | | | | | QTEYALVKEQRAEERRLKQYYS |
| | | | | | | SLTNERLEGLQQQMTAAKSPAT |
| | | | | | | KGKLNKAIRAERESIWQEAGIN |
| | | | | | | ADMMVAEYEASDRNATLDTESG |
| | | | | | | LLWPTKNTIKADVARAIKDGPR |
| | | | | | | IRGYDGGGRIGGQPSTKGISVA |
| | | | | | | EALAGTSNWLRITGPHEKCTRG |
| | | | | | | HHNRLNTAWIRIDSEGRNPVWA |
| | | | | | | VIPFVMHRPLPADAHITNVYLV |
| | | | | | | RNRVASKFKYEVHFVLAAESWV |
| | | | | | | KDDLAAEGECAIDLNWRWMDGS |
| | | | | | | LRVASWGGMDGEEGDLFLPAKV |
| | | | | | | VRMFEQVNEIKSIRDREFNIVR |
| | | | | | | DRLAAWIKSHEVPEWLQERGKF |
| | | | | | | LSRWRSQRKLAKLVLYWRDRRF |
| | | | | | | DGDDEIFAALDGTAERVQFHGK |
| | | | | | | RDKAAWNGWRNWDKHLYEWERN |
| | | | | | | LHQKAIRCRQAIYRDFAAELTR |
| | | | | | | RYHNIVMEDINWHEIGKRAEVG |
| | | | | | | EADNPTVRAKKPIGAVGELAQY |
| | | | | | | IIQRAACPEVVEPQNTTRRCPV |
| | | | | | | CRTIGKSGPALYHECKHCGRVW |
| | | | | | | DQDKKAWLNMLLAGERLDARLT |
| | | | | | | KKKRDSLRKAHREERAA |
| MG90 | 9 | MG90-14 | protein | unknown | uncultivated | MVARKTSTLPTKIWSSAAKCPA |
| Effectors | | Effector | | | organism | VSLEKRDEILSRAHAYTHRLIL |
| | | | | | | IEQSRLTEYRATRKHYAPEIEE |
| | | | | | | LQDALQDALAWRDEDAKQQAID |
| | | | | | | SARGALRTAIAKFEADQCGAAR |
| | | | | | | AEYAKRRAAACAGMGPAIKARL |
| | | | | | | NNEILVAMLAEDTWPDSWKDLA |
| | | | | | | MADRDSQDQVKRARSECGLTKG |
| | | | | | | CYLAVENAVNAAIKDSRPLAPQ |
| | | | | | | LPPWDGSGRIAVQIENNNRWAD |
| | | | | | | IVAGRNRFLRVEYTDDRPLGSC |
| | | | | | | GNSSIRCWAVFIRVGSNSDRTP |
| | | | | | | VWLEFSARLHRHPPDDAIVKWA |
| | | | | | | WIKCYKSAGREQHRFQLTFEHE |
| | | | | | | SLRWLPKPGAVGSLDLQCCHEP |
| | | | | | | VAAGVRVARWCGSDGLSGEVVV |
| | | | | | | PDDVLGNFRFAHGIRSALDGVR |
| | | | | | | AHGYRVVRKWLALGGNHVTGWR |
| | | | | | | RLSKDWRRAQMLAVCQSYVDFH |
| | | | | | | ETKSPNDSYLPLHRSMLTRDVS |
| | | | | | | AAQDRRFALWLDSWLRKERHLW |
| | | | | | | RYWRDSEARAVNLRNTLFLQAA |
| | | | | | | HELRRKYVSLSSDTSHLIANPK |
| | | | | | | MSQTERASRQAVAPGECRAIFR |
| | | | | | | EAFGLSRAPGSRGTSKAPKKRR |
| | | | | | | RSQSGA |
| MG90 | 10 | MG90-15 | protein | unknown | uncultivated | MPTKKKTYSSARKSSKVPTRNF |
| Effectors | | Effector | | | organism | VYGVGCKGEQPTPQDVIRDQLW |
| | | | | | | EAHQYKVALIRNGLARIDAENA |
| | | | | | | ILCEVSPRLGELQRQSDDLTEK |
| | | | | | | IKTMRAELKSPREDRADSPESA |
| | | | | | | AIQAVVDERKAVYEEMKKVRKD |
| | | | | | | LFALTSTYNADLNRKLEQGLLE |
| | | | | | | LAVEKDPAFAPLLDGCPEGENP |
| | | | | | | KEFKKRVKRATSIIGPRNPKRV |
| | | | | | | ALNARLTEQMLSDDSVPDTWKR |
| | | | | | | IKEVQQECHKKNLQARKDSKCF |
| | | | | | | SGTYQAVDAAVRDAFQKSYHPR |
| | | | | | | VPKENRTGKVGIQLTGERTVNG |
| | | | | | | KTVKGLPVSEVESPDNQFSLEL |
| | | | | | | LPDHRVYGARKKHNRKEYPVMA |
| | | | | | | RARICVARGVRVETLVVMHRPL |
| | | | | | | PEDGVIKWAYLEVKRVGLKTRY |
| | | | | | | ELRLVIEYFPKPRPKVTKKPLL |
| | | | | | | AVNFGYRAMADGTIRLATTWDG |
| | | | | | | EEVGGITLPSHIKRDEEFSRRL |
| | | | | | | DGHADKRENTAQKELGSWKKTV |
| | | | | | | KDMPDELKEKIRNSGQWRAHGK |
| | | | | | | LAVATHLLVRTYVPESETTVRR |
| | | | | | | LWHTWRVERLEQGLDLEDERPF |
| | | | | | | LENWFREQGVRSRNARFALYME |
| | | | | | | WWRRKDVHLVDLAKGFKARYKR |
| | | | | | | RIKEIVRVQVARWRDSYEVIAS |
| | | | | | | NEWDMRKTARKPKPENDTRTKR |
| | | | | | | EINANGVRQIGGPGIVNLYLKE |
| | | | | | | VFGDRRMTTDSKGASKIHFGCG |
| | | | | | | GEGTGIETDLRCNKCGRGEDQD |
| | | | | | | INAARHTWKEANGTLNGTPPKK |
| | | | | | | APKRKRSSRLAETTSSV |
| MG119 | 11 | MG119-60 | protein | unknown | uncultivated | MAEKQKEVIITRKIEVHLHHDP |
| Effectors | | Effector | | | organism | VADPDKVEYNRHWEVWHTINNN |
| | | | | | | LYKAANRIVSHKFENDEYEHRL |
| | | | | | | QLHSPEFQEINRKFARAKKEQL |
| | | | | | | SAEEIADLRSRIKELYAQFANE |
| | | | | | | KRTFLRGGAAVGPNPEQNSTYK |
| | | | | | | VVSDEFGKLVPSEILTCLNQSV |
| | | | | | | ASTYSEYKKQITRGERTLPNYK |
| | | | | | | KGMPVPIPMKAAGKLRLRRRDD |
| | | | | | | NSIYVLTAGGLEWDLSFGRDRS |
| | | | | | | NNREIVERVLSGQYDVGGSGIQ |
| | | | | | | EKGRKIFLLLVVKLPASQNELN |
| | | | | | | PERVVGVDLGLNIPLYAALNDN |
| | | | | | | EYGGLSIGSRDKFMKERERMSA |
| | | | | | | RRRELQRSLRISTQGGHGRSHK |
| | | | | | | LKALDQFEEKERNWVHTQNHIF |
| | | | | | | SRQVVEYAKSNGAGVIQMESLK |
| | | | | | | GFGKDKEEHIEQGFKYVVRYWS |
| | | | | | | YFELQTLIQQKAEREGIEVRKV |
| | | | | | | NPYHTSQTCSFCGHYEKGQRVS |
| | | | | | | QSTFICKNPACAHGKGKQLKDG |
| | | | | | | TFAGINADWNAARNIALSTEWV |
| | | | | | | KE |
| MG119 | 12 | MG119-82 | protein | unknown | uncultivated | MAAEFTCITRKIEVHLHRHGEG |
| Effectors | | Effector | | | organism | DEAEMRLKEEYRIWDEINDNLY |
| | | | | | | KAANRVVSHCFENDSYEYRLKL |
| | | | | | | HSPRFQEIEKLLKNAKRNKLSN |
| | | | | | | DDIRELKEERSLLESDEKKQRL |
| | | | | | | AFLQGNSGKGSEQNSTYNVISN |
| | | | | | | EFVDVIPSEVLTNLNQDISSTY |
| | | | | | | KNYALEVERGARTIPNYKKGIP |
| | | | | | | VPFSIKRNGELRLRKREDGSVY |
| | | | | | | ITFPRGLEWDLNFGRDRSNNRE |
| | | | | | | IVERVLSGLYDVGNSSIQETKS |
| | | | | | | RKRFLLLVVKIPKEASKLDTNK |
| | | | | | | VVGVDLGVAVPLYAALNDNQYG |
| | | | | | | GMSIGSYEQFTKVRQRMFAQKK |
| | | | | | | ELQRSLRHTTNGGHGRKQKLQA |
| | | | | | | LERLEGKERNWVHLQNHIFSKS |
| | | | | | | LIEYALGNKAGVVQMERLTGFG |
| | | | | | | RDVNDELGEGYKFVLRYWSFFE |
| | | | | | | LQTMIEYKAKAAGIEVRYINPY |
| | | | | | | HTSQTCSFCGHYEKGQRINQST |
| | | | | | | FICKNPDCIKGKGERKSDGSYK |
| | | | | | | GINADWNAARNIALSKEYVEKK |
| | | | | | | KK |
| MG126 | 13 | MG126-1 | protein | unknown | uncultivated | MAFKKRNDDTHYKVYRYGAILR |
| Effectors | | Effector | | | organism | PKIDGEKNPDELLIQDQLRQAN |
| | | | | | | EYYNVLVDIETDRRNEWNAIID |
| | | | | | | ADKDVIKAKSAVEEACDRVEAA |
| | | | | | | ERALKDARCKTGKRRPDRKLAQ |
| | | | | | | AVSREKRKRTKARKAVDSAKER |
| | | | | | | AKQKKKKVLDRHKMKFKKIFNE |
| | | | | | | ARSEAISKGLYWGTAAVTLDAL |
| | | | | | | PQSGPPPSKRPLYGDGQIAVQI |
| | | | | | | QCKSIPDKYFQIHDQEGWRSKR |
| | | | | | | PGKLLYIRVASKNKAGEITNHG |
| | | | | | | GHTPVWAKVPFLLDRPLPENAP |
| | | | | | | VKWIQVVKKTVGMTKHWSISFT |
| | | | | | | VELPGKRSIVEKVEKTQCRGKV |
| | | | | | | IAFDLGWRKNADGSIRVAYGTD |
| | | | | | | SSGKSFEITIPAWLSNGWDRVQ |
| | | | | | | YIRSVRDKMEDQIRDELHEWRW |
| | | | | | | GRKIPDWLTEKLKESYNWKAQK |
| | | | | | | RLGALLGHWRDHRFEGDASMVA |
| | | | | | | KIEKWDKKDRHLLKYERYHERQ |
| | | | | | | CIHQRRELYRHWARWLAKNYDA |
| | | | | | | VVFEDLNFAELYKKPDIESEKD |
| | | | | | | GVIGLHRRASPGILRATIEYAF |
| | | | | | | TDEKKRAVRVKHEGSSQTCSSC |
| | | | | | | WEITMNVGKEVKAKCEACGYEA |
| | | | | | | DRDCNASRVLLARGGVVRTSPG |
| | | | | | | RRTKPKAQWYERLMAVPLRVRE |
| | | | | | | GQEKLGRNHSQSDNQGADFVTV |
| MG126 | 14 | MG126-2 | protein | unknown | uncultivated | MYGHASDPSRVYAYGAQPPRGE |
| Effectors | | Effector | | | organism | DGDLIRAQIDAGWRYRNDLVAL |
| | | | | | | ELDRRERVGEVVRAHYPDVADQ |
| | | | | | | DAAIEETEAEIEDARAAIRAAN |
| | | | | | | QRKRARDPGPGRARTIREMRDR |
| | | | | | | LANLRREHRAARKRAYADDAVQ |
| | | | | | | ADLAVVDTGARTEQKRLRAESG |
| | | | | | | LHWGSYLAVEQSLSGIRSGAPP |
| | | | | | | RFARRATWGGQVAVQVQGGATW |
| | | | | | | EELTEGHGQFRVEVEPLPPNAT |
| | | | | | | PGGRRSKRPYCRAYFRVGSEGP |
| | | | | | | GNRTPVWAMVRFVLHRPIPDDG |
| | | | | | | HVKWVRLTRQRVGTDEKWQVQF |
| | | | | | | VVAREEWVHHDTGDASGVCGVD |
| | | | | | | LGWRTLDDGSLRVAYAMGEDGE |
| | | | | | | SEELVIPAERVGALDKADSLRA |
| | | | | | | IRDREFDLIRAQLEHWLATEPD |
| | | | | | | LPEWLAEDTRTLGQWRAQARLA |
| | | | | | | AVVIRWRDQRFEGDDDIYESIE |
| | | | | | | AWRKQDKHLYDWEGASRAKFQR |
| | | | | | | WRKQHYERFAARLRWRYETVIV |
| | | | | | | EDADWAQMARTPPAEEHDEPER |
| | | | | | | LNRRRMARIASPGYLREALEHG |
| | | | | | | HPDVRRAPSANTTRTCSVCGCD |
| | | | | | | PAGDWDPITELDYRCEAGHVMD |
| | | | | | | QDCNAAINLMHASGDVVRE |
| MG127 | 15 | MG127-1 | protein | unknown | uncultivated | MSNAIASHFASMILRAYKTELD |
| Effectors | | Effector | | | organism | PTVEQRRALARHVAGARVAHNW |
| | | | | | | VLERWRLLAGLPRLVSFLRALD |
| | | | | | | GISAAREKQAFEAWRLGFILHA |
| | | | | | | LLCGERRTIRRPKRTKKGGVTE |
| | | | | | | YTEKIVFSARPPPGPTLDQSPD |
| | | | | | | SDRIHAQLTAEKRIDESTLAWL |
| | | | | | | TSLSSFAVREGAGDVGSGYKAY |
| | | | | | | FRRLKKHLKGNHSECKPRRHRR |
| | | | | | | GCQLGEPKERSARERRYHVDQP |
| | | | | | | NPIRITARTILLPGIGEVRLKE |
| | | | | | | RGYLPETATTLDAKGHSKGSHR |
| | | | | | | LKQGGKVCGVGISERDGRWRVS |
| | | | | | | VRAEVPFPRVQPRGCGNALRWC |
| | | | | | | VVPRISGRKIGVENGVRVLVAT |
| | | | | | | YDGDTASTFVDSGLRDDERIER |
| | | | | | | FVRTRKLWERRMARRWREGVAV |
| | | | | | | RDQSTGWREAKRRVRHYHARIV |
| | | | | | | DLRDDRVGKAVRRIVDSGAEVA |
| | | | | | | LLREPHVADLLNRETAPDARVR |
| | | | | | | NRLAPDVHGARMGDVRRRLEYK |
| | | | | | | MLWARGRVERVDRFEPVTKRCS |
| | | | | | | ECGAVRATSPAYPDFRCAQCGH |
| | | | | | | VEDRDDANAPKNLFGYDSSGSS |
| | | | | | | SSGEAGPRSAGSKAPEGVSNGR |
| | | | | | | EKRSARVEGQPSATAPYRSGNR |
| | | | | | | ARPGAPISDGPIGPIFHSESEN |
| | | | | | | EQSCAAQPCAEQFRSATASPLA |
| | | | | | | DEADRSQTILQARERSTRSLDE |
| | | | | | | SKSK |
| MG90-4 | 16 | 90-4_IG2 | nucleo- | unknown | uncultivated | GCCCGTACCGAAAGCCCCCTCC |
| effector | | | tide | | organism | TGGAAGCCTACGGGTTGTTGTT |
| intergenic | | | | | | CACAAGCATGGTTGGAGGGCGA |
| region | | | | | | GGTTGAAGCGGGTGATGTGGCG |
| encoding | | | | | | GCGGAGCCTTCGTTGTCGGGGG |
| potential | | | | | | ACCTTCAGGGAGAGGGCGGTTC |
| tracrRN | | | | | | GATGTGGCAGGTGTGGGAGTAA |
| A | | | | | | GATTGGGTTGCTTCGCACTCGG |
| | | | | | | TTCTCTCAGGATGAGGTCGAGT |
| | | | | | | TGCTTTCCATCCACTTGCTTCA |
| | | | | | | CATCCGGCGGGGGTG |
| MG90-4 | 17 | 90-4_IG3 | nucleo- | unknown | uncultivated | AGTATCTACACCAGAAGAGCAA |
| effector | | | tide | | organism | GCTCGTGAGATTCTCGATGCGG |
| intergenic | | | | | | CTGGGGTGACCTACGATCGGGT |
| region | | | | | | CTATGTCTACGATGGGTGCACG |
| encoding | | | | | | CAAGAGATTTGCGGGCCCATGC |
| potential | | | | | | CCCGGGTCATTGTTCGGCGGGA |
| tracrRNA | | | | | | GGGTTCCGTTCCCGTGTGTTAC |
| | | | | | | AGTATACCTGAACTCAGGGAAG |
| | | | | | | CGGTGGGGGAGTGGAAGACGAG |
| | | | | | | GTGGGAACGAATTTCGTCGAGG |
| | | | | | | GATTGAATTTCCTTGGAGGCAG |
| | | | | | | ATCGTAGTAGGGGGTGAACGAC |
| | | | | | | GGGACGAAAGGAGGCCGAGTGG |
| | | | | | | CCTACAAAATGGTGAGACTCTT |
| | | | | | | CGATGGCTACCAGCTCAACCAC |
| | | | | | | CCGTGCGAAACGTTTCAAGGGT |
| | | | | | | GGCTCACGACCTCAAATGGTGA |
| | | | | | | GACTGGCTACTACGGCTACCGC |
| | | | | | | CGTGACGCCAAAAAGGACTCCC |
| | | | | | | GGAAGGCACGTCGTGCTAATGA |
| | | | | | | CAAGGCCGCTTGCCGGGACTAT |
| | | | | | | TGAGGCCCCCTTTTGGGGGCCT |
| | | | | | | CTTGCTGTTTGTGCCGCCACAT |
| | | | | | | CTTGCCGTCGATGCGGCCATCC |
| | | | | | | ACGGCGGGTTCGAGCTGGGAGT |
| | | | | | | AGCCGAGGGCGGGGCCCTCATC |
| | | | | | | GTAGTAGGGGTGAACAACGCAA |
| | | | | | | CGAGGTGTACGAAAGATC |
| MG90-4 | 18 | 90-4_IG4 | nucleo- | unknown | uncultivated | TTGTCCCATGTTTCAAGGGTGG |
| effector | | | tide | | organism | CTCACGACCTCAAATGGTGAGA |
| intergenic | | | | | | CTATCTGCGATCGGTCTAGTGG |
| region | | | | | | TCCTCCAGTGCCGGGGACCTCT |
| encoding | | | | | | CGCACCGCCCCCGGCTCTTTGA |
| potential | | | | | | AATCCTTGGTGTTTTACGGCGT |
| tracrRNA | | | | | | TCGGTGTTCAAGTAGGGTGCAT |
| | | | | | | CTAACGTTCGGAGATGTCCTCG |
| | | | | | | ATGAGGGGACCTCTCGCAAAGC |
| | | | | | | AGGCTCTAAGTACTCGAATCCT |
| | | | | | | GGTGGATTCCACGGGACCT |
| MG90-4 | 19 | 90-4_IG2_ | nucleo- | unknown | uncultivated | GAAGTGCCATTCCGCCTGACCT |
| effector | | adapters | tide | | organism | GCCCGTACCGAAAGCCCCCTCC |
| intergenic | | | | | | TGGAAGCCTACGGGTTGTTGTT |
| region | | | | | | CACAAGCATGGTTGGAGGGCGA |
| encoding | | | | | | GGTTGAAGCGGGTGATGTGGCG |
| potential | | | | | | GCGGAGCCTTCGTTGTCGGGGG |
| tracrRNA | | | | | | ACCTTCAGGGAGAGGGCGGTTC |
| plus | | | | | | GATGTGGCAGGTGTGGGAGTAA |
| adapters | | | | | | GATTGGGTTGCTTCGCACTCGG |
| | | | | | | TTCTCTCAGGATGAGGTCGAGT |
| | | | | | | TGCTTTCCATCCACTTGCTTCA |
| | | | | | | CATCCGGCGGGGGTGAGGCTAG |
| | | | | | | GTGGAGGCTCAGTG |
| MG90-4 | 20 | 90-4_IG3_ | nucleo- | unknown | uncultivated | GAAGTGCCATTCCGCCTGACCT |
| effector | | adapters | tide | | organism | AGTATCTACACCAGAAGAGCAA |
| intergenic | | | | | | GCTCGTGAGATTCTCGATGCGG |
| region | | | | | | CTGGGGTGACCTACGATCGGGT |
| encoding | | | | | | CTATGTCTACGATGGGTGCACG |
| potential | | | | | | CAAGAGATTTGCGGGCCCATGC |
| tracrRNA | | | | | | CCCGGGTCATTGTTCGGCGGGA |
| plus | | | | | | GGGTTCCGTTCCCGTGTGTTAC |
| adapters | | | | | | AGTATACCTGAACTCAGGGAAG |
| | | | | | | CGGTGGGGGAGTGGAAGACGAG |
| | | | | | | GTGGGAACGAATTTCGTCGAGG |
| | | | | | | GATTGAATTTCCTTGGAGGCAG |
| | | | | | | ATCGTAGTAGGGGGTGAACGAC |
| | | | | | | GGGACGAAAGGAGGCCGAGTGG |
| | | | | | | CCTACAAAATGGTGAGACTCTT |
| | | | | | | CGATGGCTACCAGCTCAACCAC |
| | | | | | | CCGTGCGAAACGTTTCAAGGGT |
| | | | | | | GGCTCACGACCTCAAATGGTGA |
| | | | | | | GACTGGCTACTACGGCTACCGC |
| | | | | | | CGTGACGCCAAAAAGGACTCCC |
| | | | | | | GGAAGGCACGTCGTGCTAATGA |
| | | | | | | CAAGGCCGCTTGCCGGGACTAT |
| | | | | | | TGAGGCCCCCTTTTGGGGGCCT |
| | | | | | | CTTGCTGTTTGTGCCGCCACAT |
| | | | | | | CTTGCCGTCGATGCGGCCATCC |
| | | | | | | ACGGCGGGTTCGAGCTGGGAGT |
| | | | | | | AGCCGAGGGCGGGGCCCTCATC |
| | | | | | | GTAGTAGGGGTGAACAACGCAA |
| | | | | | | CGAGGTGTACGAAAGATCAGGC |
| | | | | | | TAGGTGGAGGCTCAGTG |
| MG90-4 | 21 | 90-4_IG4_ | nucleo- | unknown | uncultivated | GAAGTGCCATTCCGCCTGACCT |
| effector | | adapters | tide | | organism | TTGTCCCATGTTTCAAGGGTGG |
| intergenic | | | | | | CTCACGACCTCAAATGGTGAGA |
| region | | | | | | CTATCTGCGATCGGTCTAGTGG |
| encoding | | | | | | TCCTCCAGTGCCGGGGACCTCT |
| potential | | | | | | CGCACCGCCCCCGGCTCTTTGA |
| tracrRNA | | | | | | AATCCTTGGTGTTTTACGGCGT |
| plus | | | | | | TCGGTGTTCAAGTAGGGTGCAT |
| adapters | | | | | | CTAACGTTCGGAGATGTCCTCG |
| | | | | | | ATGAGGGGACCTCTCGCAAAGC |
| | | | | | | AGGCTCTAAGTACTCGAATCCT |
| | | | | | | GGTGGATTCCACGGGACCTAGG |
| | | | | | | CTAGGTGGAGGCTCAGTG |
| MG118-2 | 22 | 118-2_5U40_ | nucleo- | unknown | uncultivated | TAATACGACTCACTATAGGCTA |
| minimal | | 38_R | tide | | organism | AAGCCAATGATGATTGCTCTTT |
| array | | | | | | ACGAAGAGACTGGAGATATCTT |
| with T7 | | | | | | GAACCTTGCATCCCCGGAAGAG |
| promoter, | | | | | | AGTCGCTAAAGCCAATGATGAT |
| two | | | | | | TGCTCTTTACGAAGAGAC |
| repeats | | | | | | |
| in the | | | | | | |
| reverse | | | | | | |
| ori- | | | | | | |
| entation, | | | | | | |
| and | | | | | | |
| one spacer | | | | | | |
| MG118-2 | 23 | 118-2_5U40_ | nucleo- | unknown | uncultivated | GAAGTGCCATTCCGCCTGACCT |
| minimal | | 38_R_ | tide | | organism | TAATACGACTCACTATAGGCTA |
| array | | adapters | | | | AAGCCAATGATGATTGCTCTTT |
| with T7 | | | | | | ACGAAGAGACTGGAGATATCTT |
| promoter, | | | | | | GAACCTTGCATCCCCGGAAGAG |
| two | | | | | | AGTCGCTAAAGCCAATGATGAT |
| repeats | | | | | | TGCTCTTTACGAAGAGACAGGC |
| in the | | | | | | TAGGTGGAGGCTCAGTG |
| reverse | | | | | | |
| ori- | | | | | | |
| entation, | | | | | | |
| and one | | | | | | |
| spacer | | | | | | |
| plus | | | | | | |
| adapters | | | | | | |
| MG90-4 | 24 | 90-4_5U67_ | nucleo- | unknown | uncultivated | TAATACGACTCACTATAGGTTT |
| minimal | | 36_F | tide | | organism | CAAGGGTGGCTCACGACCTCAA |
| array | | | | | | ATGGTGAGACGTCGAGGCTTGC |
| with T7 | | | | | | GACGTGGTGGCTCACGGAAGAC |
| promoter, | | | | | | GAGTTTCAAGGGTGGCTCACGA |
| two | | | | | | CCTCAAATGGTGAGAC |
| repeats | | | | | | |
| in the | | | | | | |
| forward | | | | | | |
| ori- | | | | | | |
| entation, | | | | | | |
| and one | | | | | | |
| spacer | | | | | | |
| MG90-4 | 25 | 90-4_5U67_ | nucleo- | unknown | uncultivated | TAATACGACTCACTATAGGTCT |
| minimal | | 36_R | tide | | organism | CACCATTTGAGGTCGTGAGCCA |
| array | | | | | | CCCTTGAAACGTCGAGGCTTGC |
| with T7 | | | | | | GACGTGGTGGCTCACGGAAGAC |
| promoter, | | | | | | GAGTCTCACCATTTGAGGTCGT |
| two | | | | | | GAGCCACCCTTGAAAC |
| repeats | | | | | | |
| in the | | | | | | |
| reverse | | | | | | |
| ori- | | | | | | |
| entation, | | | | | | |
| and one | | | | | | |
| spacer | | | | | | |
| MG90-4 | 26 | 90-4_5U67_ | nucleo- | unknown | uncultivated | GAAGTGCCATTCCGCCTGACCT |
| minimal | | 36F_ | tide | | organism | TAATACGACTCACTATAGGTTT |
| array | | adapters | | | | CAAGGGTGGCTCACGACCTCAA |
| with T7 | | | | | | ATGGTGAGACGTCGAGGCTTGC |
| promoter, | | | | | | GACGTGGTGGCTCACGGAAGAC |
| two | | | | | | GAGTTTCAAGGGTGGCTCACGA |
| repeats | | | | | | CCTCAAATGGTGAGACAGGCTA |
| in the | | | | | | GGTGGAGGCTCAGTG |
| forward | | | | | | |
| ori- | | | | | | |
| entation, | | | | | | |
| and | | | | | | |
| one | | | | | | |
| spacer | | | | | | |
| plus | | | | | | |
| adapters | | | | | | |
| MG90-4 | 27 | 90-4_5U67_ | nucleo- | unknown | uncultivated | GAAGTGCCATTCCGCCTGACCT |
| minimal | | 36_R_ | tide | | organism | TAATACGACTCACTATAGGTCT |
| array | | adapters | | | | CACCATTTGAGGTCGTGAGCCA |
| with T7 | | | | | | CCCTTGAAACGTCGAGGCTTGC |
| promoter, | | | | | | GACGTGGTGGCTCACGGAAGAC |
| two | | | | | | GAGTCTCACCATTTGAGGTCGT |
| repeats | | | | | | GAGCCACCCTTGAAACAGGCTA |
| in the | | | | | | GGTGGAGGCTCAGTG |
| reverse | | | | | | |
| ori- | | | | | | |
| entation, | | | | | | |
| and | | | | | | |
| one | | | | | | |
| spacer | | | | | | |
| plus | | | | | | |
| adapters | | | | | | |
| MG118-2 | 28 | MG118-2_U40_ | nucleo- | unknown | uncultivated | TAATACGACTCACTATAGTGAT |
| crRNA | | 18 nt | tide | | organism | GATTGCTCTTTACGAAGAGACT |
| with | | target | | | | GGAGATATCTTGAACCT |
| trimmed | | | | | | |
| repeat | | | | | | |
| and 18 nt | | | | | | |
| universal | | | | | | |
| spacer, | | | | | | |
| target | | | | | | |
| sequence | | | | | | |
| MG118-2 | 29 | MG118- | nucleo- | unknown | uncultivated | TAATACGACTCACTATAGTGAT |
| crRNA | | 2_U67_18 nt | tide | | organism | GATTGCTCTTTACGAAGAGACG |
| with | | target | | | | TCGAGGCTTGCGACGTG |
| trimmed | | | | | | |
| repeat | | | | | | |
| and 18 nt | | | | | | |
| universal | | | | | | |
| spacer, | | | | | | |
| target | | | | | | |
| sequence | | | | | | |
| MG118-2 | 30 | MG118- | nucleo- | unknown | uncultivated | UGAUGAUUGCUCUUUACGAAGA |
| crRNA | | 2_U40_18 nt | tide | | organism | GACUGGAGAUAUCUUGAACCU |
| with | | | | | | |
| trimmed | | | | | | |
| repeat | | | | | | |
| and 18 nt | | | | | | |
| universal | | | | | | |
| spacer | | | | | | |
| MG118-2 | 31 | MG118- | nucleo- | unknown | uncultivated | UGAUGAUUGCUCUUUACGAAGA |
| crRNA | | 2_U67_18 nt | tide | | organism | GACGUCGAGGCUUGCGACGUG |
| with | | | | | | |
| trimmed | | | | | | |
| repeat | | | | | | |
| and 18 nt | | | | | | |
| universal | | | | | | |
| spacer | | | | | | |
| MG90-4 | 32 | MG90- | nucleo- | unknown | uncultivated | TAATACGACTCACTATAGTGCA |
| sgRNA | | 4_sgRNA_ | tide | | organism | CCCTACTTGAACACCGAACGCC |
| with 10 | | 10 bp_RAR_ | | | | GTAAAACACCAAGGATTTCAAA |
| RAR and | | U40_24_ | | | | GAGCCGGGGGCGGTGCGAGAGG |
| 24 nt | | target | | | | TCCCCGGCACTGGAGGACCACT |
| universal | | | | | | AGACCGATCGCAGATAGTCTCA |
| spacer, | | | | | | CCATGAAAATGGTGAGACTGGA |
| target | | | | | | GATATCTTGAACCTTGCATC |
| sequence | | | | | | |
| MG90-4 | 33 | MG90- | nucleo- | unknown | uncultivated | TAATACGACTCACTATAGTGCA |
| sgRNA | | 4_sgRNA_ | tide | | organism | CCCTACTTGAACACCGAACGCC |
| with 16 | | 16bp_RAR_ | | | | GTAAAACACCAAGGATTTCAAA |
| RAR and | | U40_24_ | | | | GAGCCGGGGGCGGTGCGAGAGG |
| 24 nt | | target | | | | TCCCCGGCACTGGAGGACCACT |
| universal | | | | | | AGACCGATCGCAGATAGTCTCA |
| spacer, | | | | | | CCATTTGAGGGAAACCTCAAAT |
| target | | | | | | GGTGAGACTGGAGATATCTTGA |
| sequence | | | | | | ACCTTGCATC |
| MG90-4 | 34 | MG90- | nucleo- | unknown | uncultivated | UGCACCCUACUUGAACACCGAA |
| sgRNA | | 4_sgRNA_ | tide | | organism | CGCCGUAAAACACCAAGGAUUU |
| with 10 | | 10 bp_RAR_ | | | | CAAAGAGCCGGGGGCGGUGCGA |
| RAR and | | U40_24 | | | | GAGGUCCCCGGCACUGGAGGAC |
| 24 nt | | | | | | CACUAGACCGAUCGCAGAUAGU |
| universal | | | | | | CUCACCAUGAAAAUGGUGAGAC |
| spacer | | | | | | UGGAGAUAUCUUGAACCUUGCA |
| | | | | | | UC |
| MG90-4 | 35 | MG90- | nucleo- | unknown | uncultivated | UGCACCCUACUUGAACACCGAA |
| sgRNA | | 4_sgRNA_ | tide | | organism | CGCCGUAAAACACCAAGGAUUU |
| with 16 | | 16 bp_RAR_ | | | | CAAAGAGCCGGGGGCGGUGCGA |
| RAR and | | U40_24 | | | | GAGGUCCCCGGCACUGGAGGAC |
| 24 nt | | | | | | CACUAGACCGAUCGCAGAUAGU |
| universal | | | | | | CUCACCAUUUGAGGGAAACCUC |
| spacer | | | | | | AAAUGGUGAGACUGGAGAUAUC |
| | | | | | | UUGAACCUUGCAUC |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-99. (canceled)

100. An engineered nuclease system comprising:
(a) an endonuclease comprising a RuvCI, RuvCII, or RuvCIII domain having at least 75% sequence identity to a RuvCI, RuvCII, or RuvCIII domain of any one of SEQ ID NOs: 1-15; and
(b) an engineered guide ribonucleic acid configured to form a complex with said endonuclease, wherein said engineered guide ribonucleic acid comprises a spacer sequence configured to hybridize to a target nucleic acid sequence.

101. The engineered nuclease system of claim 100, wherein said endonuclease comprises a WED II domain having at least 75% sequence identity to a WED II domain of any one of SEQ ID NOs: 1-15.

102. The engineered nuclease system of claim 100, wherein said endonuclease is not a Cas12a endonuclease.

103. The engineered nuclease system of claim 100, wherein said endonuclease comprises an amino acid sequence having at least 75% sequence identity to any one of SEQ ID NOs: 1-15.

104. The engineered nuclease system of claim 103, wherein said endonuclease comprises the amino acid sequence of any one of SEQ ID NOs: 1-15.

105. The engineered nuclease system of claim 100, wherein said endonuclease comprises one or more nuclear localization sequences (NLSs) proximal to an N- or C-terminus of said endonuclease.

106. The engineered nuclease system of claim 105, wherein said NLS comprises an amino acid sequence having at least 80% sequence identity to any one of SEQ ID NOs: 36-51.

107. The engineered nuclease system of claim 100, wherein said engineered guide ribonucleic acid comprises a nucleic acid sequence having at least 80% sequence identity to non-degenerate the sequence of any one of SEQ ID NOs: 16-21 and 30-35.

108. The engineered nuclease system of claim 107, wherein said engineered guide ribonucleic acid comprises the nucleic acid sequence of any one of SEQ ID NOs: 16-21 and 30-35.

109. The engineered nuclease system of claim 100, further comprising a single- or double-stranded deoxyribonucleic acid repair template.

110. A method of modifying a target nucleic acid locus, said method comprising delivering to said target nucleic acid locus an engineered nuclease system comprising:
(a) an endonuclease comprising a RuvCI, RuvCII, or RuvCIII domain having at least 75% sequence identity to a RuvCI, RuvCII, or RuvCIII domain of any one of SEQ ID NOs: 1-15; and
(b) an engineered guide ribonucleic acid configured to form a complex with said endonuclease and wherein said complex modifies said target nucleic acid locus.

111. The method of claim 110, wherein said endonuclease comprises a WED II domain having at least 75% sequence identity to a WED II domain of any one of SEQ ID NOs: 1-15.

112. The method of claim 110, wherein said endonuclease is not a Cas12a endonuclease.

113. The method of claim 110, wherein said endonuclease comprises an amino acid sequence having at least 75% sequence identity to any one of SEQ ID NOs: 1-15.

114. The method of claim 113, wherein said endonuclease comprises the amino acid sequence of any one of SEQ ID NOs: 1-15.

115. The method of claim 110, wherein said endonuclease comprises one or more nuclear localization sequences (NLSs) proximal to an N- or C-terminus of said endonuclease.

116. The method of claim 110, wherein said engineered guide ribonucleic acid comprises a nucleic acid sequence having at least 80% sequence identity to non-degenerate the sequence of any one of SEQ ID NOs: 16-21 and 30-35.

117. The method of claim 116, wherein said engineered guide ribonucleic acid comprises the nucleic acid sequence of any one of SEQ ID NOs: 16-21 and 30-35.

118. The method of claim 110, wherein said target nucleic acid locus is within a cell.

119. The method of claim 118, wherein said cell is a prokaryotic cell, a bacterial cell, a eukaryotic cell, a fungal cell, a plant cell, an animal cell, a mammalian cell, a rodent cell, a primate cell, a human cell, or a primary cell.
